(19) United States
(12) Patent Application Publication
(10) Pub. No.: US 2025/0000772 A1
(43) Pub. Date: Jan. 2, 2025

(54) HYDROPHOBIC AND HYDROPHILIC MODIFIED MALEATED NATURAL OILS, SALTS, COMPOSITIONS, AND METHODS OF USE

(71) Applicant: ISP Investments LLC, Wilmington, DE (US)

(72) Inventors: Osama M. MUSA, Bedminster, NJ (US); Mousumi GHOSH, Elmwood Park, NJ (US); Petros GEBRESELASSIE, Whitehouse Station, NJ (US); Philip John OTHS, Mendham, NJ (US)

(73) Assignee: ISP Investments LLC, Wilmington, DE (US)

(21) Appl. No.: 18/703,625

(22) PCT Filed: Oct. 17, 2022
(86) PCT No.: PCT/US2022/046873

§ 371 Date: Apr. 22, 2024

Related U.S. Application Data
(60) Provisional application No. 63/274,703, filed on Nov. 2, 2021.

(51) Int. Cl. A61K 8/49; A61K 8/19; A61K 8/92; A61Q 11/00; C07C 69/604

(52) U.S. Cl. CPC A61K 8/4973; A61K 8/19; A61K 8/922; A61Q 11/00; C07C 69/604

(57) ABSTRACT

The present invention provides oral care compositions comprising a reaction product of (a) a maleated natural oil, comprising a natural oil with maleated functionality; and (b) a base. The present invention also provides oral care compositions comprising: (A) a reaction product of (a) a maleated natural oil, comprising a natural oil with maleated functionality; and (b) a base; wherein the reaction product comprises a maleated functionality partially reacted with the base; (B) reacting the reaction product from (A) with a functionalized or unfunctionalized moiety selected from the group consisting of hydrophobic moieties, hydrophilic moieties, and combinations thereof. The oral care compositions can be used in a wide variety of applications.

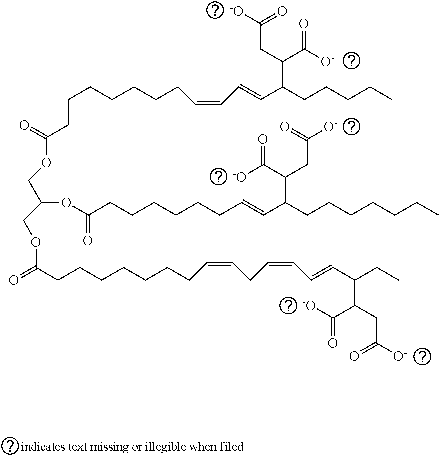

### FIELD OF THE INVENTION

The present invention provides oral care compositions comprising a reaction product of (a) a maleated natural oil, comprising a natural oil with maleated functionality; and (b) a base. The present invention also provides oral care compositions comprising: (A) a reaction product of (a) a maleated natural oil, comprising a natural oil with maleated functionality; and (b) a base; wherein the reaction product comprises a maleated functionality partially reacted with the base; and (B) reacting the reaction product from (A) with a functionalized or unfunctionalized moiety selected from the group consisting of hydrophobic moieties, hydrophilic moieties, and combinations thereof. The oral care compositions can be used in a wide variety of applications.

### BACKGROUND OF THE INVENTION

The modified natural oils can be non-dispersible in water or alcohols. As a result, these modified natural oils can be incorporated into a wide variety of compositions. Such compositions include, but are not limited to, personal care (e.g., hair care, sun care, skin care, oral care), adhesives, coatings, paints, electronics, household, industrial and institutional (HI&I) compositions, inks, membranes, metal working fluids, oilfield chemicals, plastics and plasticizers, textiles, industrial products, biocides, pharmaceuticals/nutritionals, and agrochemical compositions.

Natural oils, such as soybean and linseed oils, are one of the most promising raw materials for the synthesis of renewable compounds, including polymers, plastics, and plasticizers. These natural materials are inexpensive, highly abundant, come from reliable and sustainable sources, and have high potential for modification. Natural oils are generally a blend of different triglycerides, the esterification products of fatty acids and glycerol, and contain varying degrees of unsaturation (i.e., double bonds). Oils can be characterized by a hydroxyl value and the fatty acid compositions. Both natural fatty acids and natural oils must be chemically modified to make them sufficiently reactive to allow structural alterations and polymerizations to occur because the olefin functional groups are relatively unreactive. Unsaturated double bonds in these compounds have been converted to epoxide functional groups and succinic anhydride functional groups, allowing the addition of many hydroxyl containing species to be introduced into the natural oils.

Maleated natural oils are natural oils that have been chemically functionalized by the chemical addition of epoxide (oxirane) and succinic anhydride functional groups. Examples include epoxidized and maleated soybean oil and linseed oil. Unsaturated natural oils lend themselves to these chemical functionalizations.

U.S. Pat. No. 9,809,538B2 describes a modified natural compound synthesized from epoxidized natural fatty acid, maleated natural fatty acid, epoxidized natural oil, or maleated natural oil; and lactam compound with hydroxyl(s) to form e.g. adhesive or beverage composition.

A discussion of reaction scheme for the maleinization reaction in the vegetable oil is provided in the article “Maleated soybean oil and its multifunctional properties,” by Gripp, Anna A., Steinberg, David C., published in Cosmetics Exhibition & Conference Proceedings, Barcelona, Mar. 22-24, 1994.

A discussion of reaction scheme for the maleinization reaction in the vegetable oil is provided in the article “Microwave Assisted Syntheses of Vegetable Oil Based Monomer,” by Rafael T. Alarcon et al., published in Journal of Polymers and the Environment 28:1265-1278, 2020.

A discussion of generalized reaction between maleic anhydride and unsaturated vegetable oils is provided in the handbook of maleic Anhydride based materials-syntheses, properties and applications by Osama M. Musa in chapter 3 page 166, published in Springer International Publishing Switzerland 2016.

U.S. Pat. No. 2,754,306A describes a reaction with a soybean oil, maleic anhydride based and iso-octyl alcohol to provide an improved plasticizer for nitrocellulose compositions.

PCT application No. 2019113068A1 and 2005071050A1 describes a technology related to metalworking fluids comprising maleated soybean oil derivatives.

A discussion of maleic anhydride polymerization and modified plant oils with polyols is provided in the article “Polymerization of Maleic Anhydride-Modified Plant Oils with Polyols,” by Tarik Eren, Selim H. Kusefoglu, Richard Wool published in Journal of Applied Polymer Science, Barcelona, Volume 90, Issue 1, Pages 197-202, 2003.

EP U.S. Pat. No. 2,754,306A describes an adhesive containing a polycondensate and a dienophile modified fatty acid as a cross-linking agent.

PCT application No. 2005071050A1 describes a metalworking fluid comprising oil in water emulsion from a reaction product of maleic anhydride and a triglyceride oil from a plant or land animal and further reacted with water, Group IA and IIA metals, ammonium hydroxide, various amines, alkanolamines, polyols, alkoxylated alkanolamines, poly(alkylene oxide)s, or polyamines or mixtures.

Mineral oils and vegetable oils are often used as components of oral care compositions. Accordingly, there is a need for materials that are renewable, natural, and biodegradable having different and controllable chemical, physical, and/or mechanical properties such that the limitations found in natural and maleated natural oils are minimized or eliminated.

Natural essential oils have been used in oral care products in recent years to provide a wide range of benefits. Conventionally, herbal based products, such as essential oils, have been included in oral care products for flavor as well as cleansing, freshening, and in some cases anti-microbial benefits. However, a need still exists for natural oil compositions that can provide improved anti-microbial benefits, and in particular natural oil compositions which eliminate microbials within the oral cavity.

In addition, each time a new active component is released to the market, it necessitates the development of a unique composition to get the active ingredient into the field. Therefore, there is a need to develop new, functionalized maleated natural oils for use oral care is essential.

To promote oral health, a variety of substances have been utilized individually and in combination. It would be highly desirable to combine the substances for optimal improvement of oral health. Accordingly, the preparation of modified maleated natural oils and their compositions described in this application comprise (i) a reaction product of (a) a maleated natural oil; and (b) one or more bases; (ii) one or more orally acceptable excipients; and (iii) one or more oral care active ingredients in an oral care composition.

### SUMMARY OF THE INVENTION

The present invention provides oral care compositions comprising a reaction product of (a) a maleated natural oil, comprising a natural oil with maleated functionality; and (b) a base. The present invention also provides oral care compositions comprising: (A) a reaction product of (a) a maleated natural oil, comprising a natural oil with maleated functionality; and (b) a base;

wherein the reaction product comprises a maleated functionality partially reacted with the base;
and (B) reacting the reaction product from (A) with a functionalized or unfunctionalized moiety selected from the group consisting of hydrophobic moieties, hydrophilic moieties, and combinations thereof. The oral care compositions can be used in a wide variety of applications.

An important aspect of the present application is to provide the above oral care compositions with an orally acceptable excipient and an oral care active ingredient.

In one aspect, the present application provides an oral care composition comprising: (a) about 0.1 to about 99.9 wt. % of at least one reaction product of a maleated natural oil and a base; (b) about 0.1 to about 99.9 wt. % of an orally acceptable excipient; and (c) about 0.1 to about 50 wt. % of an oral care active ingredient.

In another aspect, the present application provides an oral care composition comprising (a) about 0.1 to about 99.9 wt. % of a reaction product of maleated soybean oil and a base selected from the group consisting of sodium hydroxide, potassium hydroxide and calcium hydroxide; (b) about 0.1 to about 99.9 wt. % of an orally acceptable excipient; and (c) about 0.1 to about 50 wt. % of an oral care active ingredient.

In another aspect, the present invention provides an oral care composition for providing oral health benefits comprising (a) about 0.1 to about 99.9 wt. % of the reaction product of a maleated soybean oil with a base selected from the group consisting of sodium hydroxide, potassium hydroxide and calcium hydroxide; (b) about 0.1 to about 99.9 wt. % of an orally acceptable excipient; and (c) about 0.1 to about 50 wt. % of an oral care active ingredient.

### DETAILED DESCRIPTION OF THE INVENTION

The present invention provides oral care compositions comprising a reaction product of (a) a maleated natural oil, comprising a natural oil with maleated functionality; and (b) a base. The present invention also provides oral care compositions comprising: (A) a reaction product of (a) a maleated natural oil, comprising a natural oil with maleated functionality; and (b) a base; wherein the reaction product comprises a maleated functionality partially reacted with the base; and (B) reacting the reaction product from (A) with a functionalized or unfunctionalized moiety selected from the group consisting of hydrophobic moieties, hydrophilic moieties, and combinations thereof. The oral care compositions can be used in a wide variety of applications.

The disclosed and/or claimed inventive concept(s) is not limited in its application to the details of construction and the arrangement of the components or steps or methodologies set forth in the following description or illustrated in the drawings. The disclosed and/or claimed inventive concept(s) is capable of other aspects or of being practiced or carried out in various ways. Also, the phraseology and terminology employed herein is for the purpose of description and should not be regarded as limiting.

As utilized in accordance with the disclosure, the following terms, unless otherwise indicated, shall be understood to have the following meanings.

Unless otherwise defined herein, technical terms used in connection with the disclosed and/or claimed inventive concept(s) shall have the meanings that are commonly understood by those of ordinary skill in the art. Further, unless otherwise required by context, singular terms shall include pluralities and plural terms shall include the singular.

The singular forms “a,” “an,” and “the” include plural forms unless the context clearly dictates otherwise specified or clearly implied to the contrary by the context in which the reference is made. The term “Comprising” and “Comprises of” includes the more restrictive claims such as “Consisting essentially of” and “Consisting of”.

For purposes of the following detailed description, other than in any operating examples, or where otherwise indicated, numbers that express, for example, quantities of ingredients used in the specification and claims are to be understood as being modified in all instances by the term “about”. The numerical parameters set forth in the specification and attached claims are approximations that may vary depending upon the desired properties to be obtained in carrying out the invention.

All percentages, parts, proportions and ratios as used herein, are by weight of the total composition, unless otherwise specified. All such weights as they pertain to listed ingredients are based on the active level and, therefore; do not include solvents or by-products that may be included in commercially available materials, unless otherwise specified.

All publications, articles, papers, patents, patent publications, and other references cited herein are hereby incorporated herein in their entirety for all purposes to the extent consistent with the disclosure herein.

The use of the term “at least one” will be understood to include one as well as any quantity more than one, including but not limited to, 1, 2, 3, 4, 5, 10, 15, 20, 30, 40, 50, 100, etc. The term “at least one” may extend up to 100 or 1000 or more depending on the term to which it is attached. In addition, the quantities of 100/1000 are not to be considered limiting as lower or higher limits may also produce satisfactory results.

As used herein, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “each independently selected from the group consisting of” means when a group appears more than once in a structure, that group may be selected independently each time it appears.

The term “polymer” refers to a compound comprising repeating structural units (monomers) connected by covalent chemical bonds. Polymers may be further derivatized, crosslinked, grafted or end-capped. Non-limiting examples of polymers include copolymers, terpolymers, tetrapolymers, quaternary polymers, and homologues. The term “copolymer” refers to a polymer consisting essentially of two or more different types of monomers polymerized to obtain said copolymer.

The term “reaction product” refers to a substance produced from a chemical reaction of one or more reactant substances.

The term “reaction product” selected from the group of structures, comprising a maleated functionality fully or partially reacted with the base, consisting of the following structures:

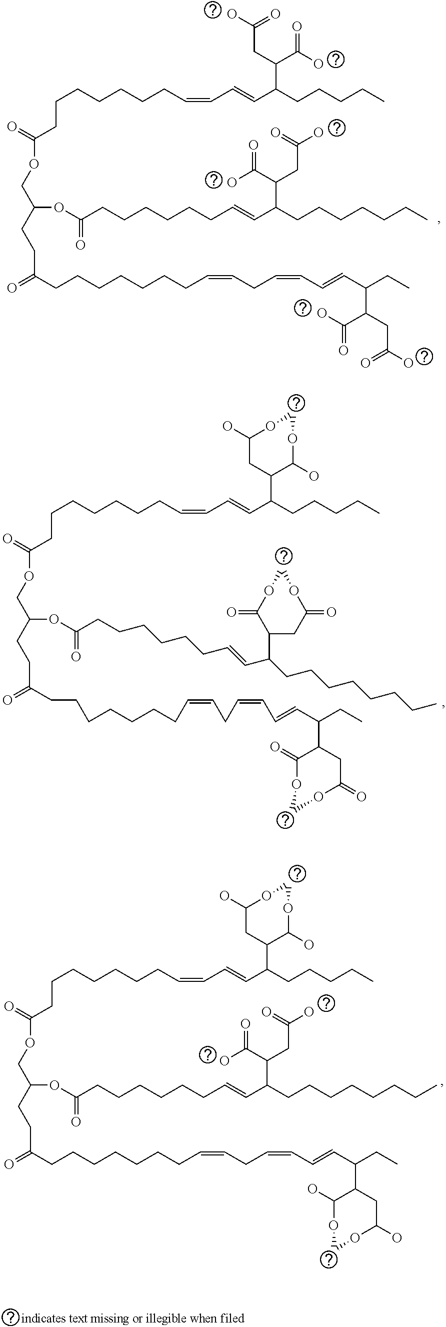

and mixtures thereof, refers to fully or partially reacted succinic anhydride structures which may comprise succinic acid and any combination of alkali metals and alkaline earth metals, in any combination.

The reaction product may be selected from the group of structures represented by the following structures:

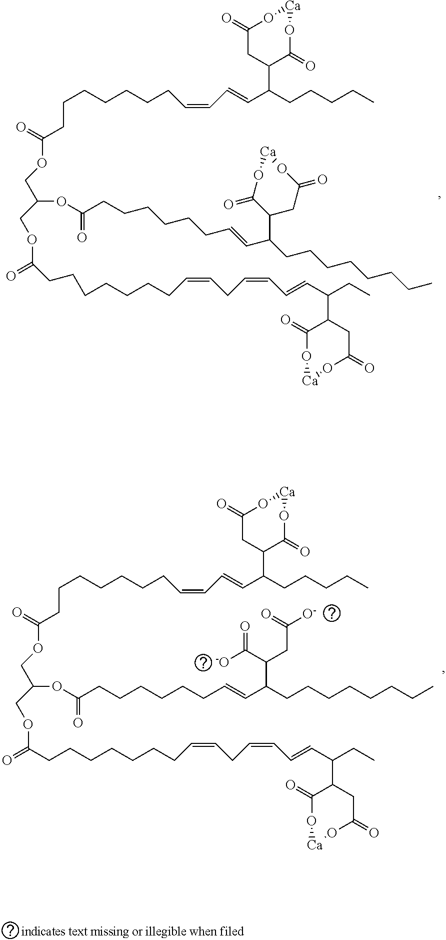

and combinations thereof, refers to fully or partially reacted succinic anhydride structures which may comprise succinic acid and any combination of alkali metals and alkaline earth metals, in any combination.

The term “natural oil” refers to compounds comprising triglycerides and may contain varying levels of fatty acids, monoglycerides, diglycerides and triglycerides refer to oil derived from plants or animal sources. Natural oils also include fatty acid glyceryl esters, which are synthesized by reacting glycerol with 1, 2, or 3 molar equivalents of a fatty acid or a mixture of fatty acids. These compounds can be mono, di or triglycerides of a single fatty acid or a mixture of fatty acids.

The term “maleated natural oil”, as used herein, refers to natural oil contains at least one or more maleated functionalities. Accordingly, the term “maleation” or “maleated” as used hereafter should be understood to mean “functionalization” insofar as the use of functionalizing reagents other than maleic anhydride are contemplated for use in the process of the invention.

The term “base”, as used herein, refers to any substance which can alter the pH of a solution from a neutral pH of 7.0 to a basic pH (i.e., 7.1 to 14). Typically, a base is a substance of a large class of compounds with one or more of the following properties: bitter taste, slippery feeling in solution, ability to turn litmus blue and to cause other indicators to take on characteristic colors, ability to react with (neutralize) acids to form salts includes both organic base or an inorganic base consisting of oxides, hydroxides, carbonates and bicarbonates of alkali metals (Group I), alkaline earth metals (Group II), transition metals and combinations thereof.

The term “alkali metal base” includes oxides, hydroxides, carbonates or bicarbonates of sodium (Na), potassium (K), rubidium (Rb), cesium (Cs), and francium (Fr).

The term “alkali-earth metal base” includes oxides, hydroxides, carbonates or bicarbonates of beryllium (Be), magnesium (Mg), calcium (Ca), strontium (Sr), barium (Ba), and radium (Ra).

The term “transition metal base” includes oxides, hydroxides, carbonates or bicarbonates of scandium (Sc), titanium (Ti), vanadium (V), chromium (Cr), manganese (Mn), iron (Fe), cobalt (Co), nickel (Ni), copper (Cu), zinc (Zn), yttrium (Y), zirconium (Zr), niobium (Nb), molybdenum (Mo), technetium (Tc), ruthenium (Ru), rhodium (Rh), palladium (Pd), silver (Ag), cadmium (Cd), hafnium (Hf), tantalum (Ta), tungsten (W), rhenium (Re), osmium (Os), iridium (Ir), platinum (Pt), gold (Au), mercury (Hg), rutherfordium (Rf), dubnium (Db), seaborgium (Sg), bohrium (Bh), hassium (Hs), meitnerium (Mt), darmstadtium (Ds) and roentgenium (Rg).

The term “orally acceptable excipients or carrier” refers to safe and effective materials and conventional additives used in oral care compositions.

As used herein, the term “oral care actives” includes bactericidal agents, natural and synthetic agents used in dentistry to inhibit bacterial growth.

As used herein, the term “moiety” or “moieties” refers to a part or a functional group(s) of a molecule.

The term “functionalized” with reference to any moiety refers to the presence of one or more functional groups in the moiety. Various functional groups may be introduced in a moiety by way of one or more functionalization reactions known to a person having ordinary skill in the art. Non-limiting examples of functionalization reactions include: alkylation, epoxidation, sulfonation, hydrolysis, amidation, esterification, hydroxylation, dihydroxylation, amination, ammonolysis, acylation, nitration, oxidation, dehydration, elimination, hydration, dehydrogenation, hydrogenation, acetalization, halogenation, dehydrohalogenation, Michael addition, aldol condensation, Canizzaro reaction, Mannich reaction, Clasien condensation, Suzuki coupling, and the like. In one non-limiting embodiment, the term “functionalized” with reference to any moiety refers to the presence of one more functional groups selected from the group consisting of alkyl, alkenyl, hydroxyl, carboxyl, halogen, alkoxy, amino, imino, and combinations thereof, in the moiety.

As used herein, the term “hydrophilic” means that the compound has an affinity for water, whereas “hydrophobic” means not having an affinity for water.

The terms are relative terms, where a hydrophilic moiety it has a higher affinity for water than a hydrophobic moiety, but the hydrophilic moiety may or may not be completely water soluble. Likewise, hydrophobic moieties have less of an affinity for water than hydrophilic moieties, but the hydrophobic moieties may not necessarily be water-repellant. While hydrophilic moieties have an affinity for water and other polar solvents, hydrophobic moieties tend to have an affinity for oils, fats, and other non-polar solvents.

The term “unreacted maleated functionality” refers to a composition comprising a reaction product of maleated natural oil in which the components of maleated functionality are completely unreacted and properties are not changed.

The term “hydrocarbyl” includes straight-chain and branched-chain alkyl, alkenyl, alkynyl, cycloalkyl, cycloalkenyl, aryl groups, and combinations thereof with optional heteroatom(s). A hydrocarbyl group may be mono-, di- or polyvalent and have carbon chains contain at least 2 carbon atoms and preferably 2 to 100 carbon atoms.

The term “organic base” refers to an organic compound which acts as a base. Organic bases are usually, but not always, proton acceptors. Organic bases usually contain nitrogen atoms, which can easily be protonated. For example, amines or nitrogen-containing heterocyclic compounds have a lone pair of electrons on the nitrogen atom and can thus act as proton acceptors. Examples include pyridine, alkanamines, such as methylamine, imidazole, benzimidazole, histidine, guanidine, phosphazene bases, hydroxides of quaternary ammonium cations, and other organic cations.

The term “alkyl” refers to a functionalized or unfunctionalized, monovalent, straight-chain, branched-chain, or cyclic C₁-C₆₀ hydrocarbyl group optionally having one or more heteroatoms. In one non-limiting embodiment, an alkyl is a C₁-C₄₅ hydrocarbyl group. In another non-limiting embodiment, an alkyl is a C_{1-C30} hydrocarbyl group. Non-limiting examples of alkyl include methyl, ethyl, n-propyl, isopropyl, n-butyl, isobutyl, tert-butyl, n-pentyl, isopentyl, n-hexyl, n-heptyl, n-octyl, 2-ethylhexyl, tert-octyl, iso-norbornyl, n-dodecyl, tert-dodecyl, n-tetradecyl, n-hexadecyl, n-octadecyl, n-eicosyl, cyclobutyl, cyclopentyl, cyclohexyl, and the like. The definition of “alkyl” also includes groups obtained by combinations of straight-chain, branched-chain and/or cyclic structures.

The term “aryl” refers to a functionalized or unfunctionalized, monovalent, aromatic hydrocarbyl group optionally having one or more heteroatoms. The definition of aryl includes carbocyclic and heterocyclic aromatic groups. Non-limiting examples of aryl groups include phenyl, naphthyl, indenyl, indanyl, azulenyl, fluorenyl, anthracenyl, furyl, thienyl, pyridyl, pyrrolyl, oxazolyl, thiazolyl, imidazolyl, pyrazolyl, 2-pyrazolinyl, pyrazolidinyl, isoxazolyl, isothiazolyl, 1,2,3-oxadiazolyl, 1,2,3-triazolyl, 1,3,4-thiadiazolyl, pyridazinyl, pyrimidinyl, pyrazinyl, 1,3,5-triazinyl, 1,3,5-trithianyl, indolizinyl, indolyl, isoindolyl, 3H-indolyl, indolinyl, benzo[b]furanyl, 2,3-dihydrobenzofuranyl, benzo[b]thiophenyl, 1H-indazolyl, benzimidazolyl, benzthiazolyl, purinyl, 4H-quinolizinyl, isoquinolinyl, cinnolinyl, phthalazinyl, quinazolinyl, quinoxalinyl, 1,8-naphthridinyl, pteridinyl, carbazolyl, acridinyl, phenazinyl, phenothiazinyl, phenoxyazinyl, pyrazolo[1,5-c]triazinyl, and the like.

The term “aralkyl” refers to an alkyl group comprising one or more aryl substituent(s) wherein “aryl” and “alkyl” are as defined above. Non-limiting examples of aralkyl groups include benzyl, 2-phenyl-ethyl, 3-phenyl-propyl, 4-phenyl-butyl, 5-phenyl-pentyl, 4-phenylcyclohexyl, 4-benzylcyclohexyl, 4-phenylcyclohexylmethyl, 4-benzylcyclohexylmethyl, and the like.

The term “alkylene” refers to a functionalized or unfunctionalized, divalent, straight-chain, branched-chain, or cyclic C₁-C₄₀ hydrocarbyl group optionally having one or more heteroatoms. In one non-limiting embodiment, an alkylene is a C₁-C₃₀ group. In another non-limiting embodiment, an alkylene is a C₁-C₂₀ group. Non-limiting examples of alkylene groups include:

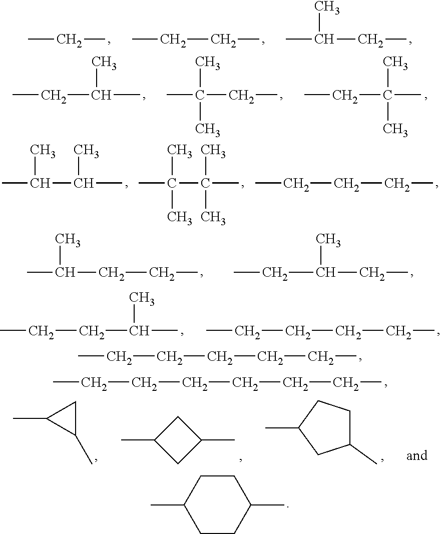

The term “arylene” refers to a functionalized or unfunctionalized, divalent, aromatic hydrocarbyl group optionally having one or more heteroatoms. The definition of arylene includes carbocyclic and heterocyclic groups. Non-limiting examples of arylene groups include phenylene, naphthylene, pyridinylene, and the like.

The term “heteroatom” refers to oxygen, nitrogen, sulfur, silicon, phosphorous, or halogen. The heteroatom(s) may be present as a part of one or more heteroatom-containing functional groups. Non-limiting examples of heteroatom-containing functional groups include ether, hydroxy, epoxy, carbonyl, carboxamide, carboxylic ester, carboxylic acid, imine, imide, amine, sulfonic, sulfonamide, phosphonic, and silane groups. The heteroatom(s) may also be present as a part of a ring such as in heteroaryl and heteroarylene groups.

The present invention provides an oral care composition comprising a reaction product of (a) a maleated natural oil, comprising a natural oil with maleated functionality; and (b) a base. Preferably, the reaction product comprises a maleated functionality fully or partially reacted with the base.

The base may be selected from the group consisting of inorganic bases, organic bases, and mixtures thereof. Preferably, the inorganic base is selected from the group consisting of oxides of alkali metals and alkaline earth metals, hydroxides of alkali metals and alkaline earth metals, carbonates of alkali metals and alkaline earth metals, bicarbonates of alkali metals and alkaline earth metals, oxides of transition metals, and combinations thereof. Preferably, the organic base is selected from the group consisting of ammonia, primary amines, secondary amines, pyridine, imidazole, benzimidazole, histidine, guanidine, and mixtures thereof. Preferably, the alkali metal is selected from the group consisting of lithium, sodium, potassium, rubidium, cesium, francium, and mixtures thereof. Preferably, the alkaline earth metal is selected from the group consisting of beryllium, magnesium, calcium, strontium, barium, radium, and mixtures thereof. Preferably, the transition metal is selected from the group consisting of oxides, hydroxides, carbonates or bicarbonates of scandium, titanium, vanadium, chromium, manganese, iron, cobalt, nickel, copper, zinc, yttrium zirconium, niobium, molybdenum, technetium, ruthenium, rhodium, palladium, silver, cadmium, hafnium, tantalum, tungsten, rhenium, osmium, iridium, platinum, gold, mercury, rutherfordium, dubnium, seaborgium, bohrium, hassium, meitnerium, darmstadtium, roentgenium, and mixtures thereof. Preferably, the inorganic base is selected from the group consisting of oxides, hydroxides, carbonates and bicarbonates of sodium, calcium, and combinations thereof.

Preferably, the maleated natural oil is selected from the group consisting of maleated avocado oils, maleated coconut oils, maleated corn oils, maleated cottonseed oils, maleated jojoba oils, maleated linseed oils, maleated nut oils, maleated olive oils, maleated palm oils, maleated raisin oils, maleated rapeseed oils, maleated safflower oils, maleated sesame oils, maleated soybean oils, maleated squash oils, maleated sunflower oils, maleated almond oils, maleated canola oils, maleated flaxseed oils, maleated grapeseed oils, maleated palm oils, maleated palm kernel oils, maleated peanut oils, maleated walnut oils, and mixture thereof. Preferably, the maleated natural oil is a maleated soybean oil and the base is sodium hydroxide.

Preferably, the reaction product is selected from the group of structures, comprising a maleated functionality fully or partially reacted with the base, consisting of the following structures:

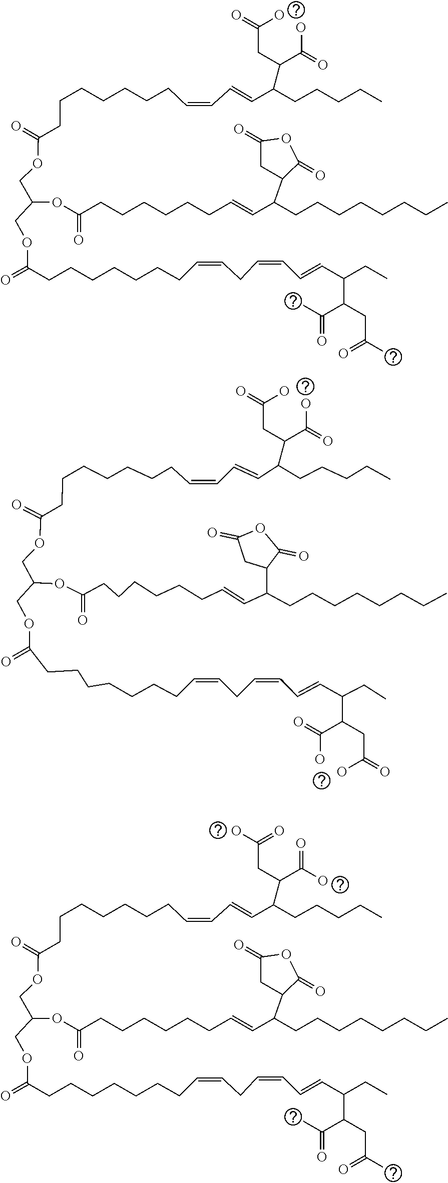 [SMILES: CCCCCCCCC(/C=C/CCCCCCC(=O)OC(COC(=O)CCCCCCC/C=C\C=C\C(CCCCC)C(CC(=O)O)C(=O)O)COC(=O)CCCCCCC/C=C\C/C=C\C=C\C(CC)C(CC(C)=O)C(C)=O)C1CC(=O)OC1=O ; CCCCCCCCC(/C=C/CCCCCCC(=O)OC(COC(=O)CCCCCCC/C=C\C=C\C(CCCCC)C(CC(=O)O)C(=O)O)COC(=O)CCCCCCC/C=C\C/C=C\C(C)=C\C(CC)C(CC(=O)O)C(=O)O)C1CC(=O)OC1=O ; CCCCCCCCC(/C=C/CCCCCCC(=O)OC(COC(=O)CCCCCCC/C=C\C=C\C(CCCCC)C(CC(=O)O)C(=O)O)COC(=O)CCCCCCC/C=C\C/C=C\C=C\C(CC)C(CC(C)=O)C(C)=O)C1CC(=O)OC1=O]

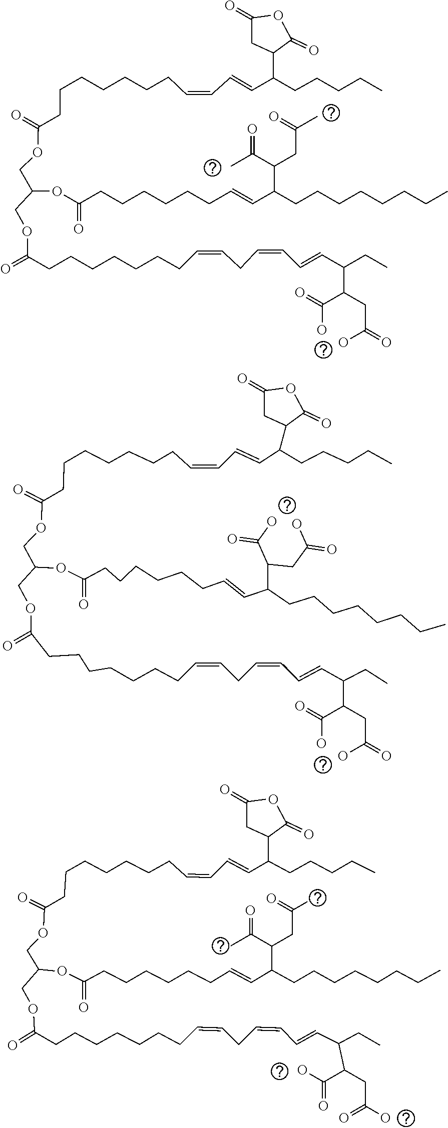 [SMILES: CCCCCCCCC(/C=C/CCCCCCC(=O)OC(COC(=O)CCCCCCC/C=C\C=C\C(CCCCC)C1CC(=O)OC1=O)COC(=O)CCCCCCC/C=C\C/C=C\C=C\C(CC)C(CC(=O)O)C(=O)O)C(CC(C)=O)C(C)=O ; CCCCCCCCC(/C=C/CCCCCCC(=O)OC(COC(=O)CCCCCCC/C=C\C=C\C(CCCCC)C1CC(=O)OC1=O)COC(=O)CCCCCCC/C=C\C/C=C\C(C)=C\C(CC)C(CC(=O)O)C(=O)O)C(CC(=O)O)C(=O)O ; CCCCCCCCC(/C=C/CCCCCCC(=O)OC(COC(=O)CCCCCCC/C=C\C=C\C(CCCCC)C1CC(=O)OC1=O)COC(=O)CCCCCCC/C=C\C/C=C\C=C\C(CC)C(CC(=O)O)C(=O)O)C(CC(C)=O)C(C)=O]

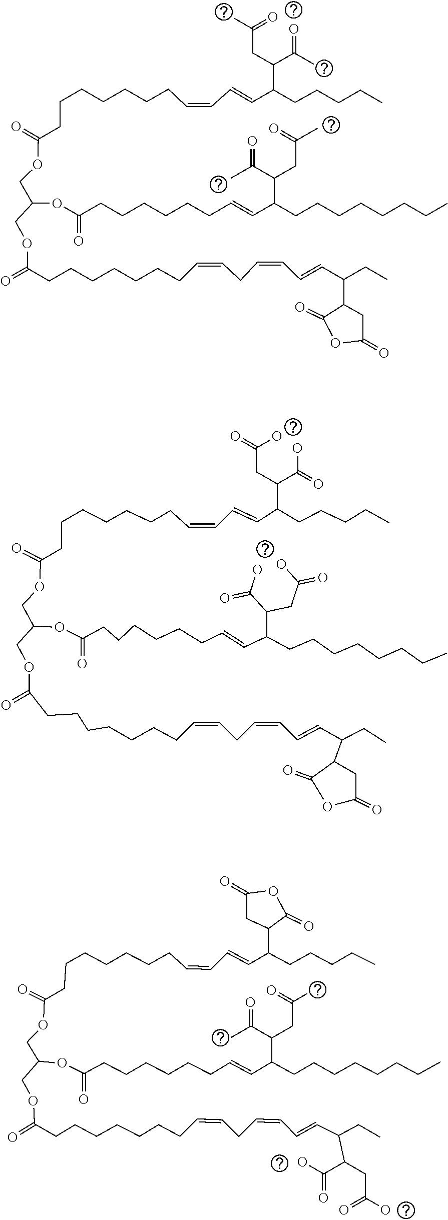 [SMILES: CCCCCCCCC(/C=C/CCCCCCC(=O)OC(COC(=O)CCCCCCC/C=C\C=C\C(CCCCC)C(CC(C)=O)C(C)=O)COC(=O)CCCCCCC/C=C\C/C=C\C=C\C(CC)C1CC(=O)OC1=O)C(CC(C)=O)C(C)=O ; CCCCCCCCC(/C=C/CCCCCCC(=O)OC(COC(=O)CCCCCCC/C=C\C=C\C(CCCCC)C(CC(=O)O)C(=O)O)COC(=O)CCCCCCC/C=C\C/C=C\C(C)=C\C(CC)C1CC(=O)OC1=O)C(CC(=O)O)C(=O)O ; CCCCCCCCC(/C=C/CCCCCCC(=O)OC(COC(=O)CCCCCCC/C=C\C=C\C(CCCCC)C1CC(=O)OC1=O)COC(=O)CCCCCCC/C=C\C/C=C\C=C\C(CC)C(CC(=O)O)C(=O)O)C(CC(C)=O)C(C)=O]

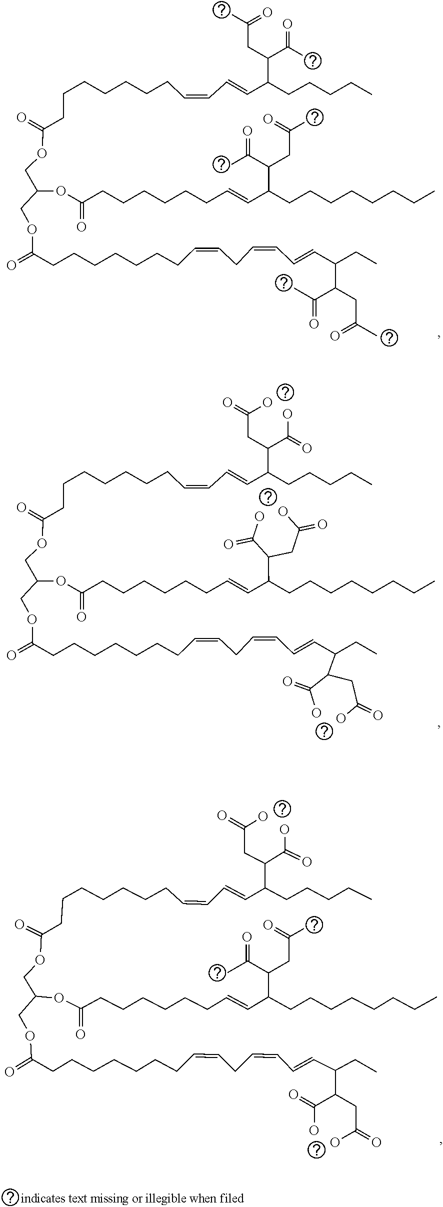 [SMILES: CCCCCCCCC(/C=C/CCCCCCC(=O)OC(COC(=O)CCCCCCC/C=C\C=C\C(CCCCC)C(CC(C)=O)C(C)=O)COC(=O)CCCCCCC/C=C\C/C=C\C=C\C(CC)C(CC(C)=O)C(C)=O)C(CC(C)=O)C(C)=O ; CCCCCCCCC(/C=C/CCCCCCC(=O)OC(COC(=O)CCCCCCC/C=C\C=C\C(CCCCC)C(CC(=O)O)C(=O)O)COC(=O)CCCCCCC/C=C\C/C=C\C=C\C(CC)C(CC(=O)O)C(=O)O)C(CC(=O)O)C(=O)O ; CCCCCCCCC(/C=C/CCCCCCC(=O)OC(COC(=O)CCCCCCC/C=C\C=C\C(CCCCC)C(CC(=O)O)C(=O)O)COC(=O)CCCCCCC/C=C\C/C=C\C=C\C(CC)C(CC(=O)O)C(=O)O)C(CC(C)=O)C(C)=O]

and mixtures thereof.

The reaction product above refers to fully or partially reacted succinic anhydride structures which may comprise succinic acid and any combination of alkali metals and alkaline earth metals, in any combination.

The oral care composition may further comprise an orally acceptable excipient and an oral care active ingredient. Preferably, reaction product comprises (A) (a) from about 0.1 to about 99.9 wt. % of a maleated natural oil, comprising a natural oil with maleated functionality; and (b) a base, and (B) (a) from about 0.1 to about 99.9 wt. % of an orally acceptable excipient; and (b) from about 0.1 to about 50 wt. % of an oral care active ingredient. Preferably, the reaction product comprises (A) (a) from about 0.1 to about 99.9 wt. % of a maleated soybean natural oil, comprising a soybean oil with maleated functionality; and (b) a base selected from the group consisting of sodium hydroxide, potassium hydroxide, calcium hydroxide; and mixtures thereof; (B) (a) from about 0.1 to about 99.9 wt. % of an orally acceptable excipient; and (b) from about 0.1 to about 50 wt. % of an oral care active ingredient.

The present invention further provides an oral care composition comprising (A) a reaction product of (a) a maleated natural oil, comprising a natural oil with maleated functionality; and (b) a base; wherein the reaction product comprises a maleated functionality partially reacted with the base; and (B) reacting the reaction product from (A) with: a functionalized or unfunctionalized moiety selected from the group consisting of hydrophobic moieties, hydrophilic moieties, and combinations thereof.

Preferably, the hydrophobic moiety is a moiety selected from the group consisting of unsubstituted or substituted alkyl, cycloalkyl, alkenyl, and aryl alcohols, wherein any of the beforehand mentioned groups may be with or without heteroatoms, containing from about C₆ to about C₃₆ atoms, unsubstituted or substituted alkyl, cycloalkyl, alkenyl, and aryl amines, wherein any of the beforehand mentioned groups may be with or without heteroatoms, containing from about C₆ to about C₃₆ atoms, silicon-based compounds, and combinations thereof.

Preferably, the hydrophilic moiety is a moiety selected from the group consisting of unsubstituted or substituted alkyl, cycloalkyl, alkenyl, and aryl alcohols, wherein any of the beforehand mentioned groups may be with or without heteroatoms, containing from about C₁ to about C₅ atoms, unsubstituted or substituted alkyl, cycloalkyl, alkenyl, and aryl amines, wherein any of the beforehand mentioned groups may be with or without heteroatoms, containing from about C₁ to about C₅ atoms, unsubstituted or substituted polyols, wherein any of the beforehand mentioned groups may be with or without heteroatoms, containing from about C₂ to about C₃₆ atoms, silanes, and combinations thereof.

Preferably, the silane is functionalized with an alcohol or an amine, and combinations thereof. Preferably, the hydrophobic alcohol is selected from the group consisting of heptanol, nonanol, decanol, dodecanol, phenol, ethylbenzyl alcohol, 2-ethyl-1-hexanol, 1-octanol, 2-octanol, 2-octyl-1-dodecyl alcohol, 2-tertradecanol, 2-hexadecanol, 3,7-dimethyl-1-octanol, 2-propyl-1-pentanol, 4-methyl-1-pentanol, and mixtures thereof. Preferably, the hydrophilic alcohol is selected from the group consisting of methanol, ethanol, propanol, isopropanol, butanol, ethylene glycol, propylene glycol, diethylene glycol, dipropylene glycol, dibutylene glycol, polyethylene glycol, methoxypolyethylene glycol, polypropylene glycol, hexylene glycol, sorbitol, neopentylglycol, eythritol, mannitol, xylitol, threitol, pentaerythritol, beta-cyclodextrin, L-Ribose, 2-deoxy-D-galactose, and mixtures thereof. Preferably, the hydrophobic amine is selected from the group consisting of benzylamine, cyclohexylamine, hexylamine, methylhexylamine, phenethylamine, octylamine, oleylamine, decylamine, dodecylamine, octadecylamine, undecyl amine, pentadecyl amine, 2-methyl butyl amine, dimethyl amine, and mixtures thereof. Preferably, hydrophilic amine is selected from the group consisting of 2-methylpentane-1,5-diamine, diethanol amine, diisopropanolamine, serinol hydrochloride, 2-amino-2-ethyl-1,3-propanediol, n-methyl-D-glucamine, D-galactosamine hydrochloride, d-glucosamine hydrochloride, D-mannosamine hydrochloride, and mixtures thereof. Preferably, the silicon based compound is a hydrophobic compound selected from the group consisting of aminopropylmethylsiloxane-dimethylsiloxane, N-ethylaminoisobutyl terminated polydimethylsiloxane, poly(1,1-dimethylsilazane) telomer, aminopropyl terminated polydimethylsiloxane, monoaminopropyl terminated polydimethylsiloxane, (tetramethylpiperidinyloxy)propylmethylsiloxane]-dimethylsiloxane copolymer, polydimethylsiloxane, carbinol (hydroxyl) terminated polydimethylsiloxane, monocarbinol terminated polydimethylsiloxane, monocarbinol terminated functional polydimethylsiloxane, [Bis(hydroxyethyl)amine] terminated polydimethylsiloxane, silanol terminated polydimethylsiloxane, silanol terminated polydiphenylsiloxane, dodecylmethylsiloxane-hydroxypolyalkyleneoxypropyl methylsiloxane, and mixtures thereof. Preferably, the silane is a hydrophilic compound selected from the group consisting of 3-aminopropylsilanetriol, N-(2-aminoethyl)-3-aminopropylsilanetriol, and mixtures thereof.

Preferably, the maleated natural oil is selected from the group consisting of maleated avocado oils, maleated coconut oils, maleated corn oils, maleated cottonseed oils, maleated jojoba oils, maleated linseed oils, maleated nut oils, maleated olive oils, maleated palm oils, maleated raisin oils, maleated rapeseed oils, maleated safflower oils, maleated sesame oils, maleated soybean oils, maleated squash oils, maleated sunflower oils, maleated almond oils, maleated canola oils, maleated flaxseed oils, maleated grapeseed oils, maleated palm oils, maleated palm kernel oils, maleated peanut oils, maleated walnut oils, and mixtures thereof. More preferably, the maleated natural oil is a maleated soybean oil.

Preferably, the reaction product is selected from the group of structures represented by the following structures:

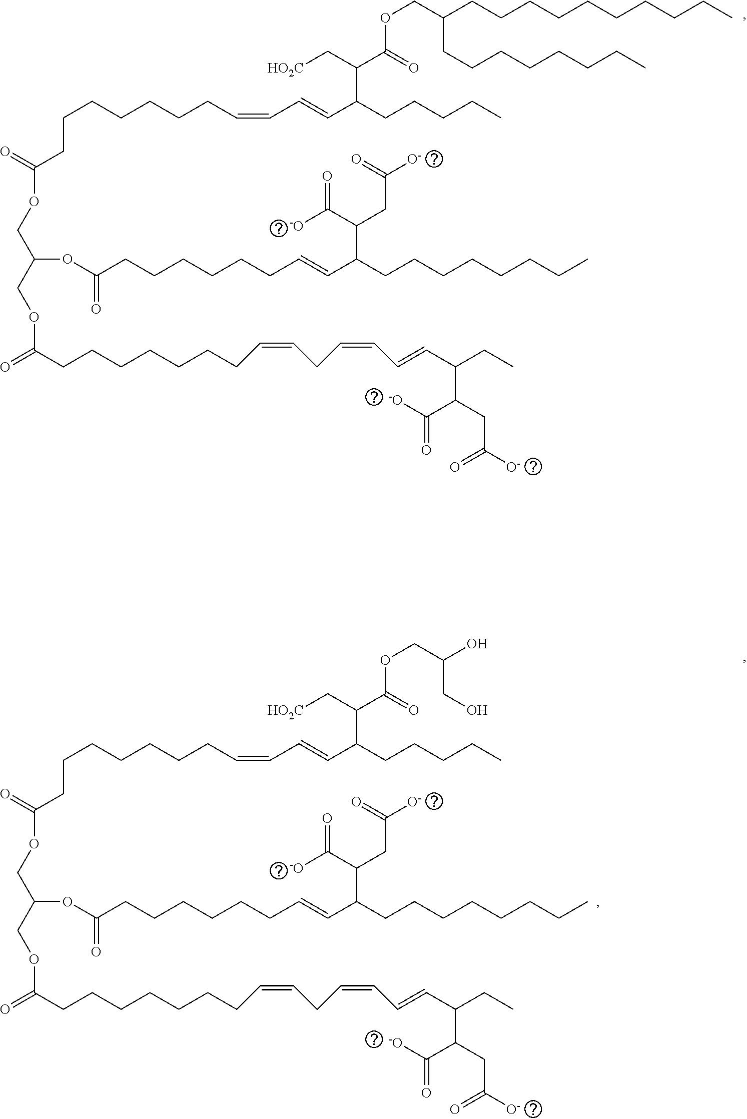

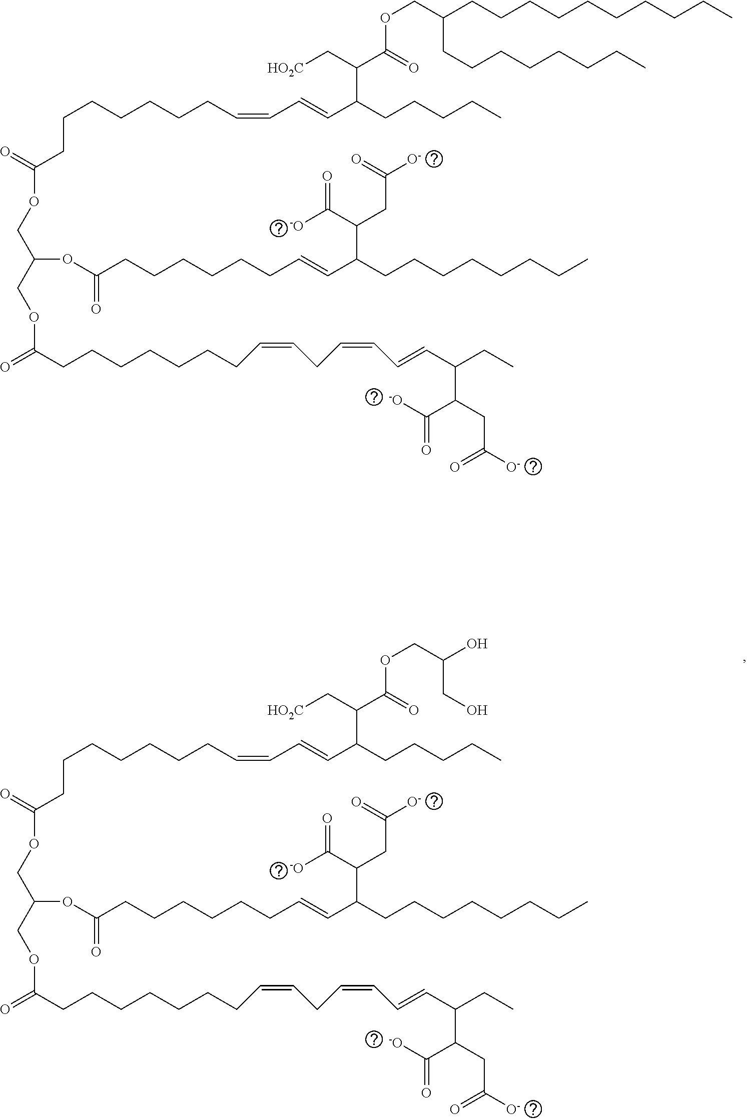

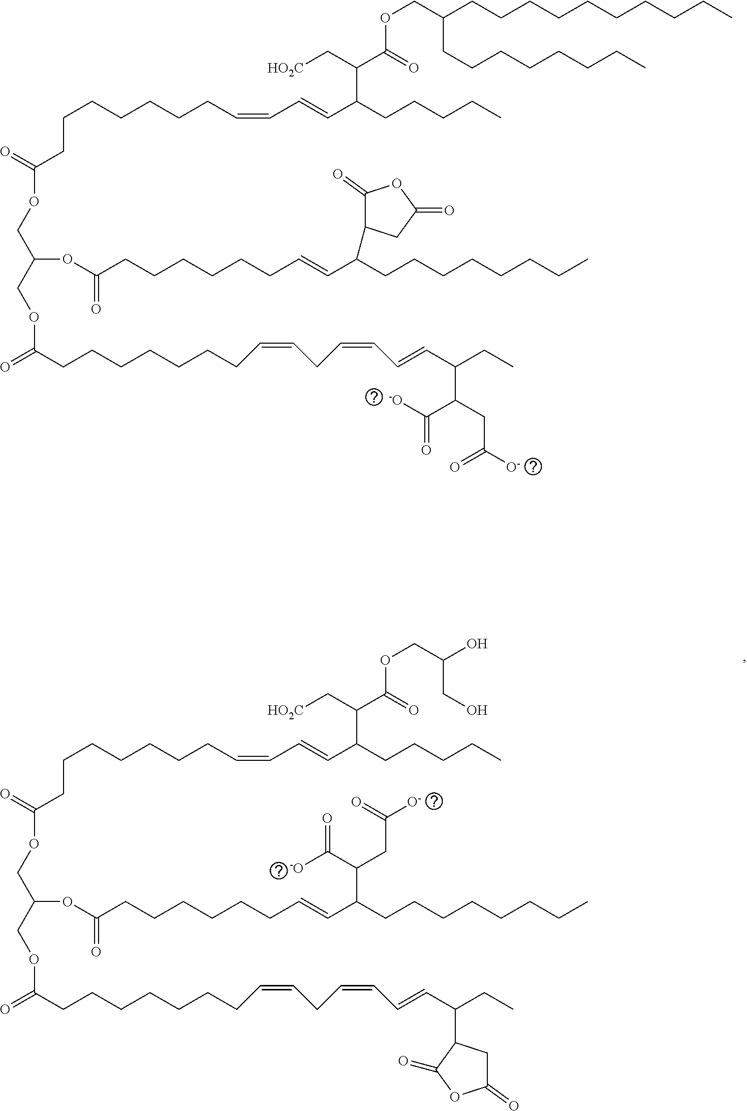

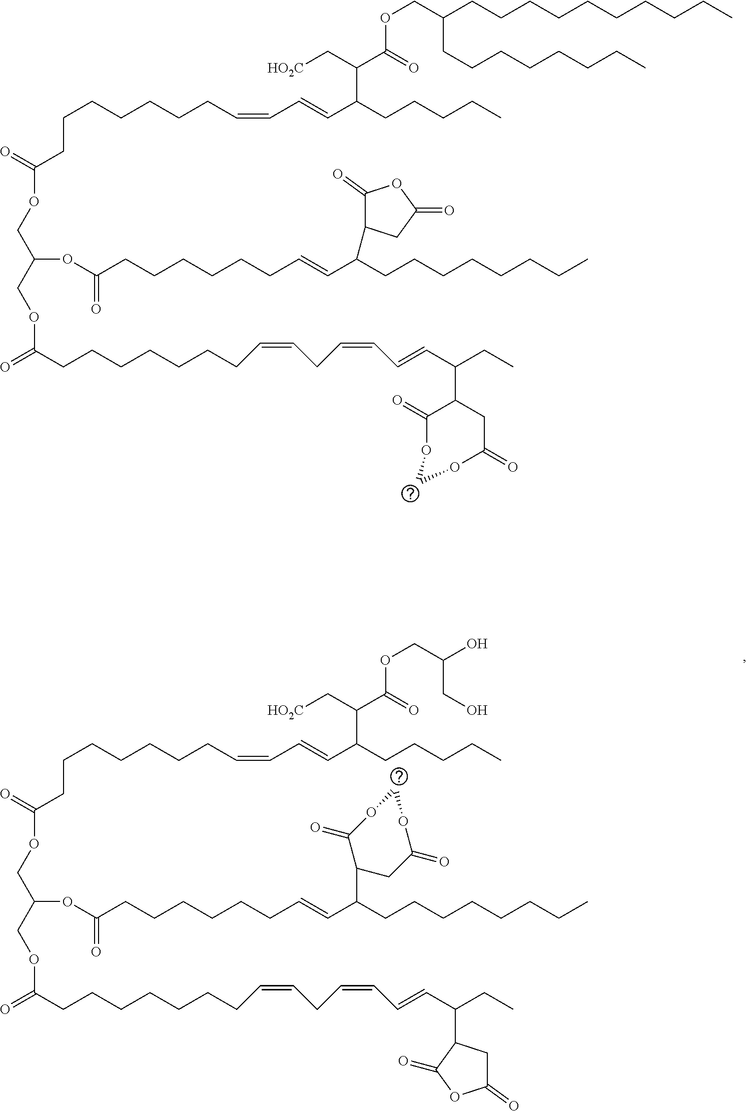

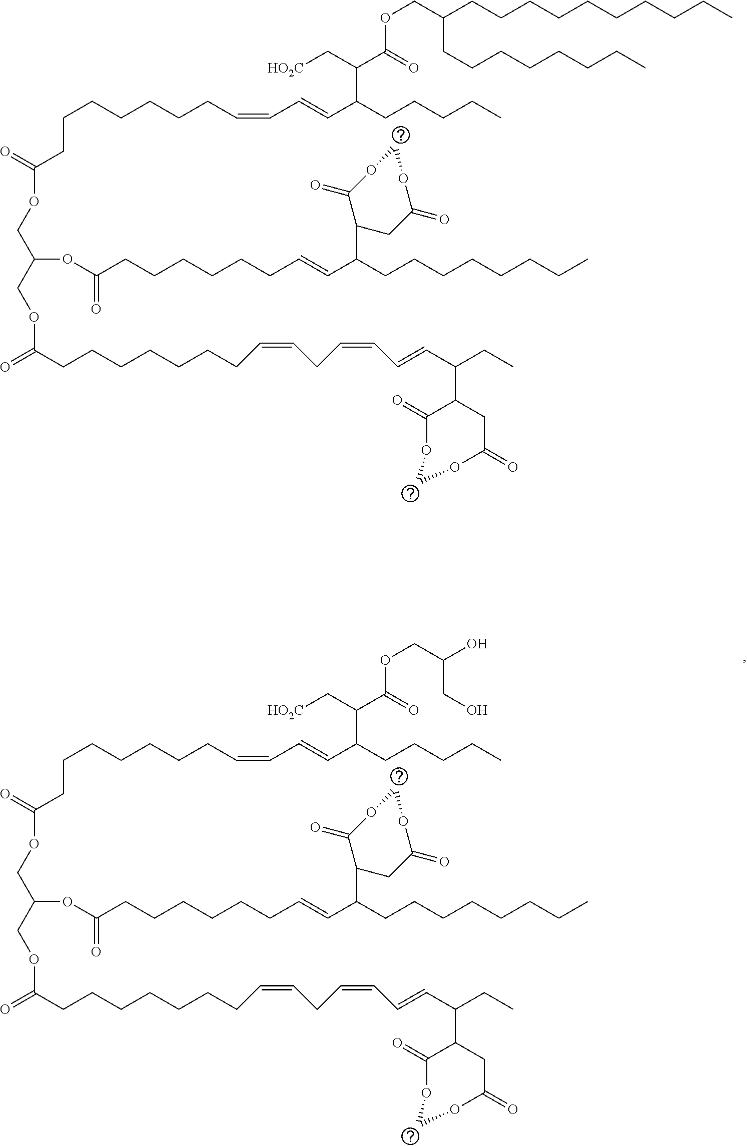

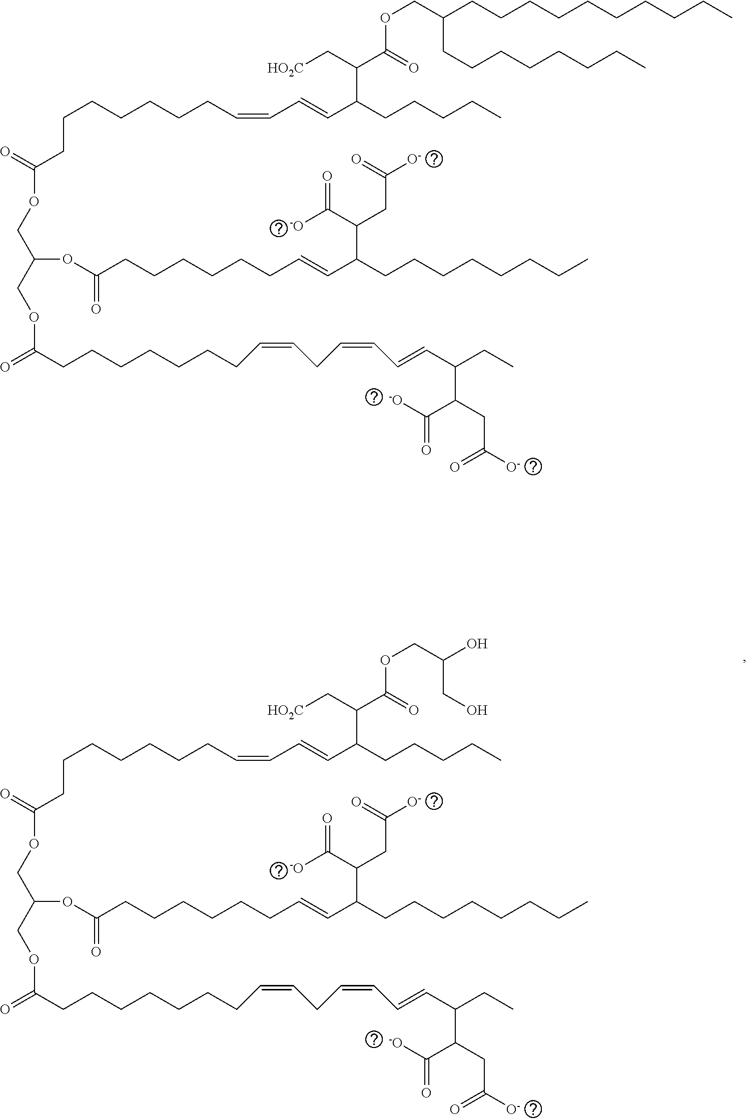

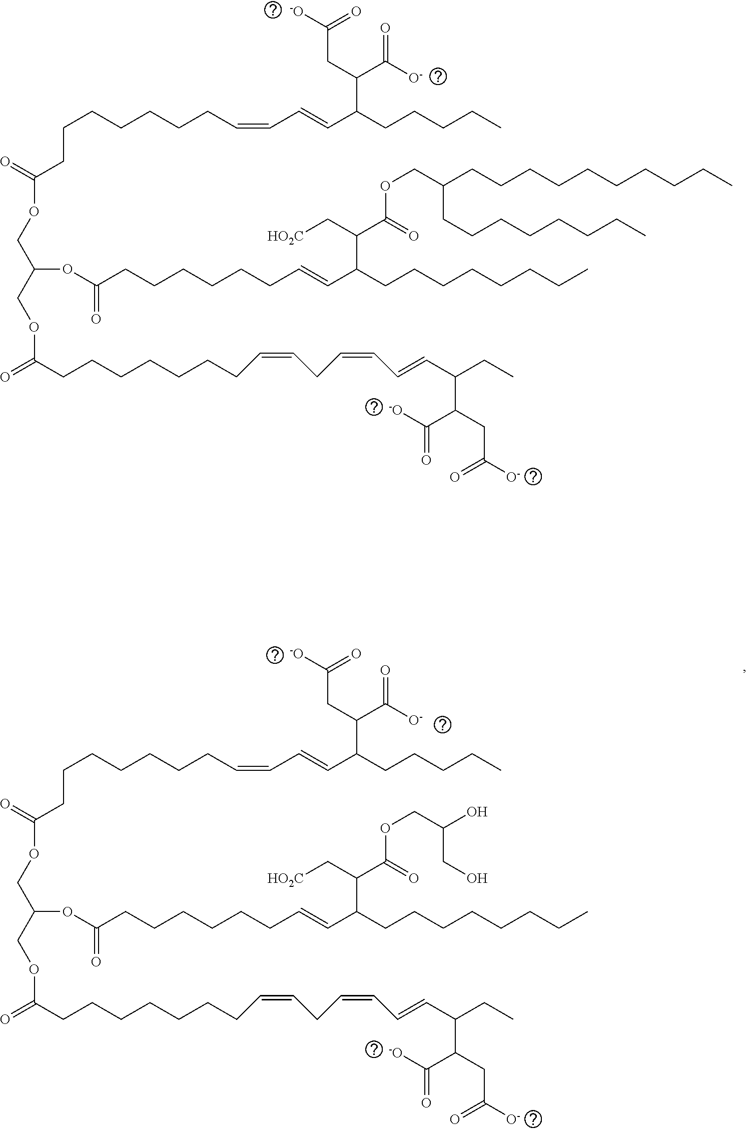

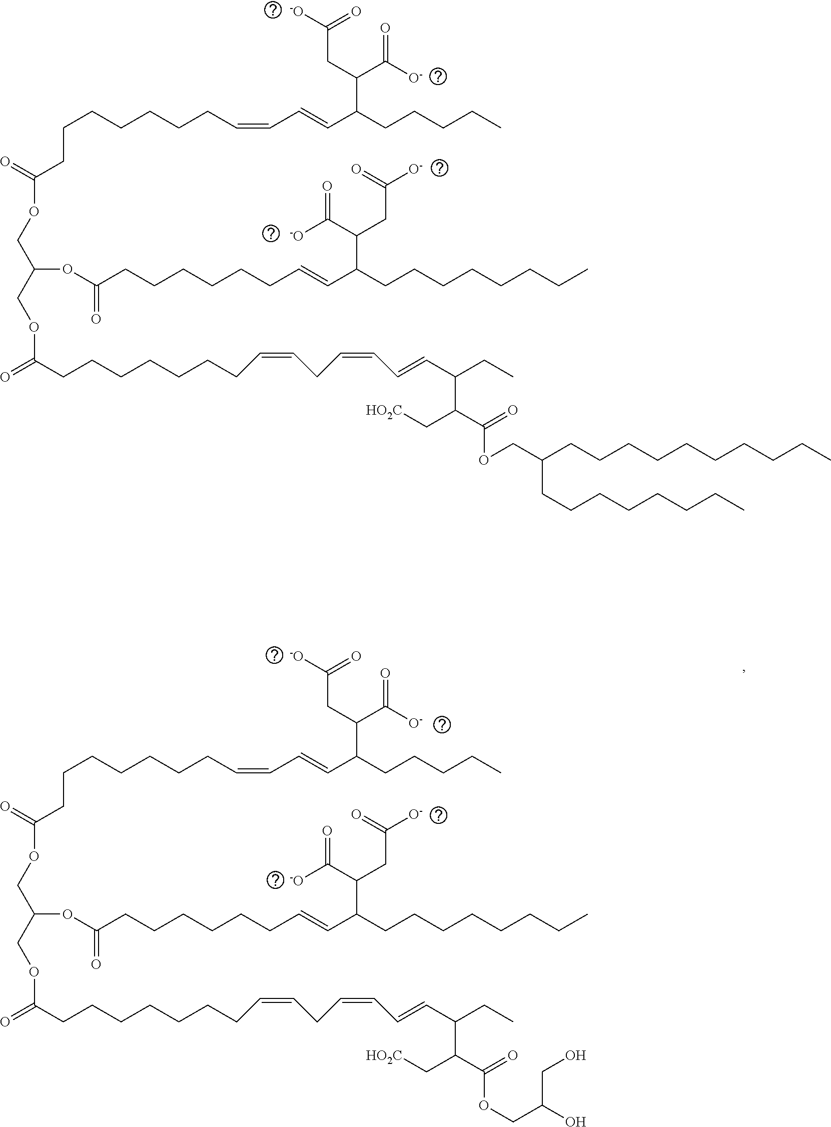

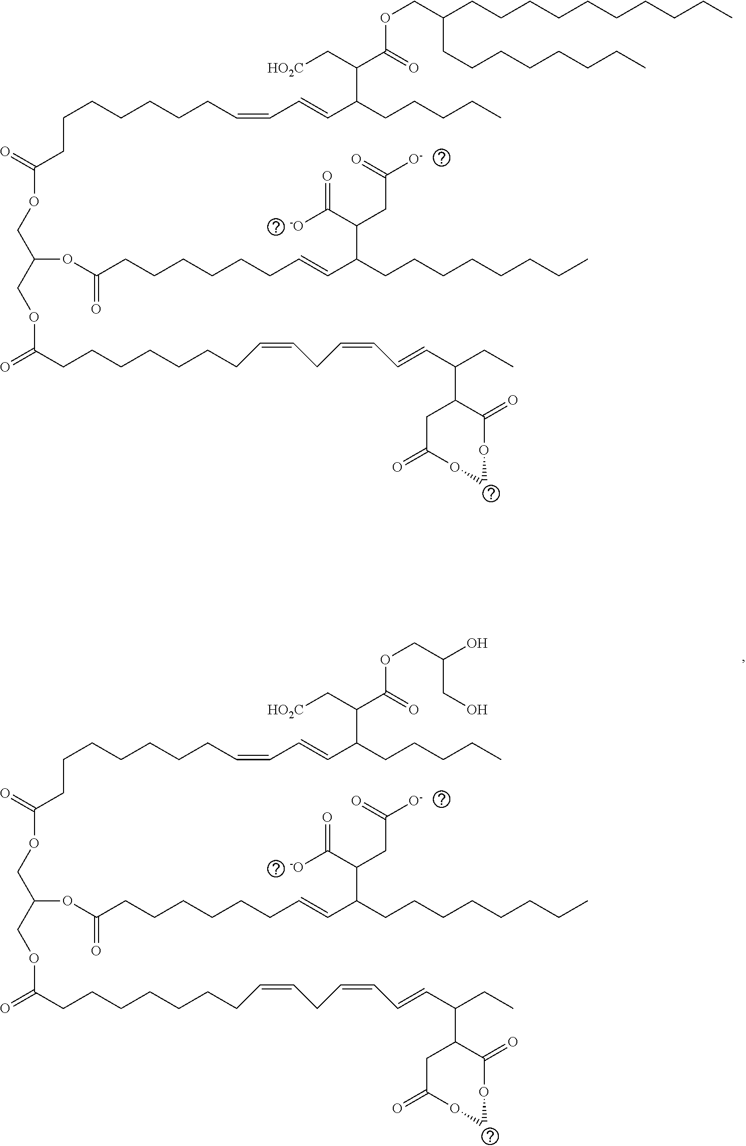

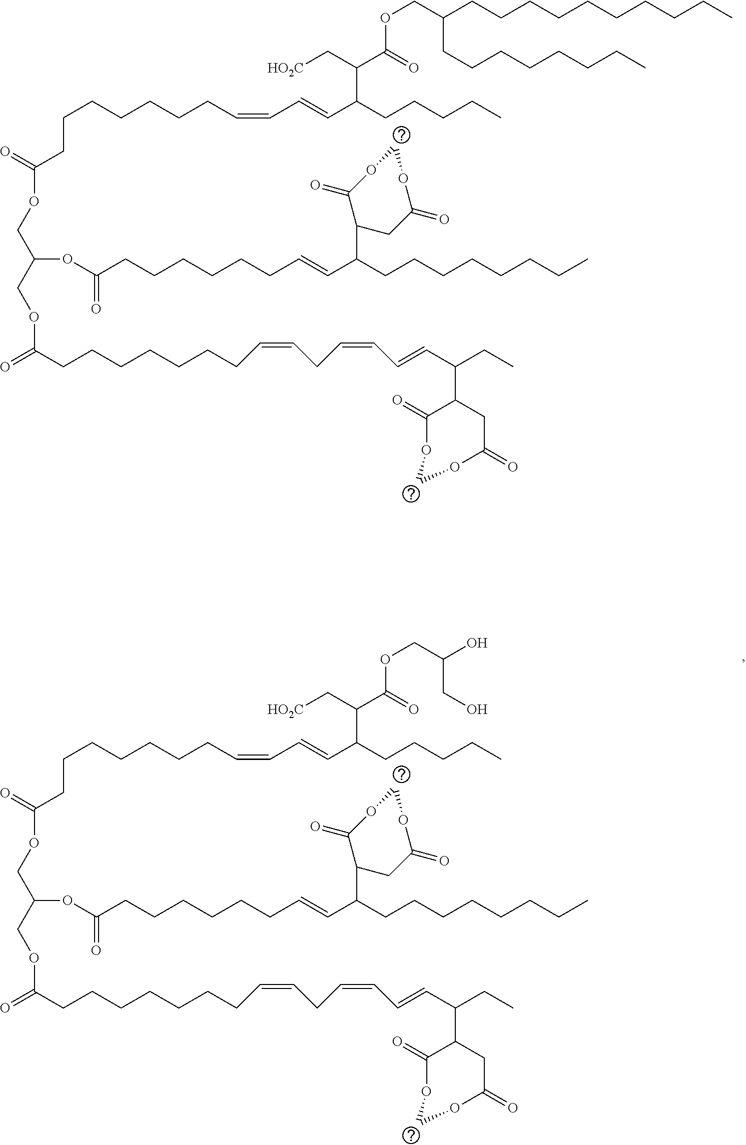

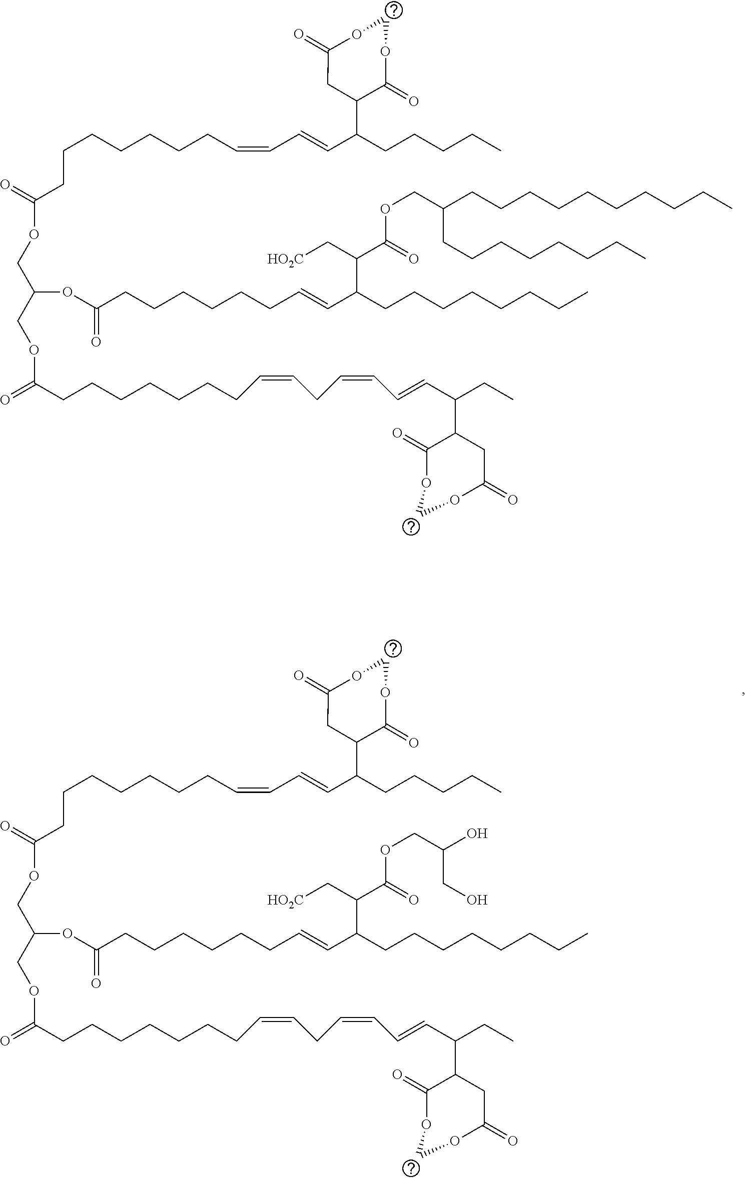

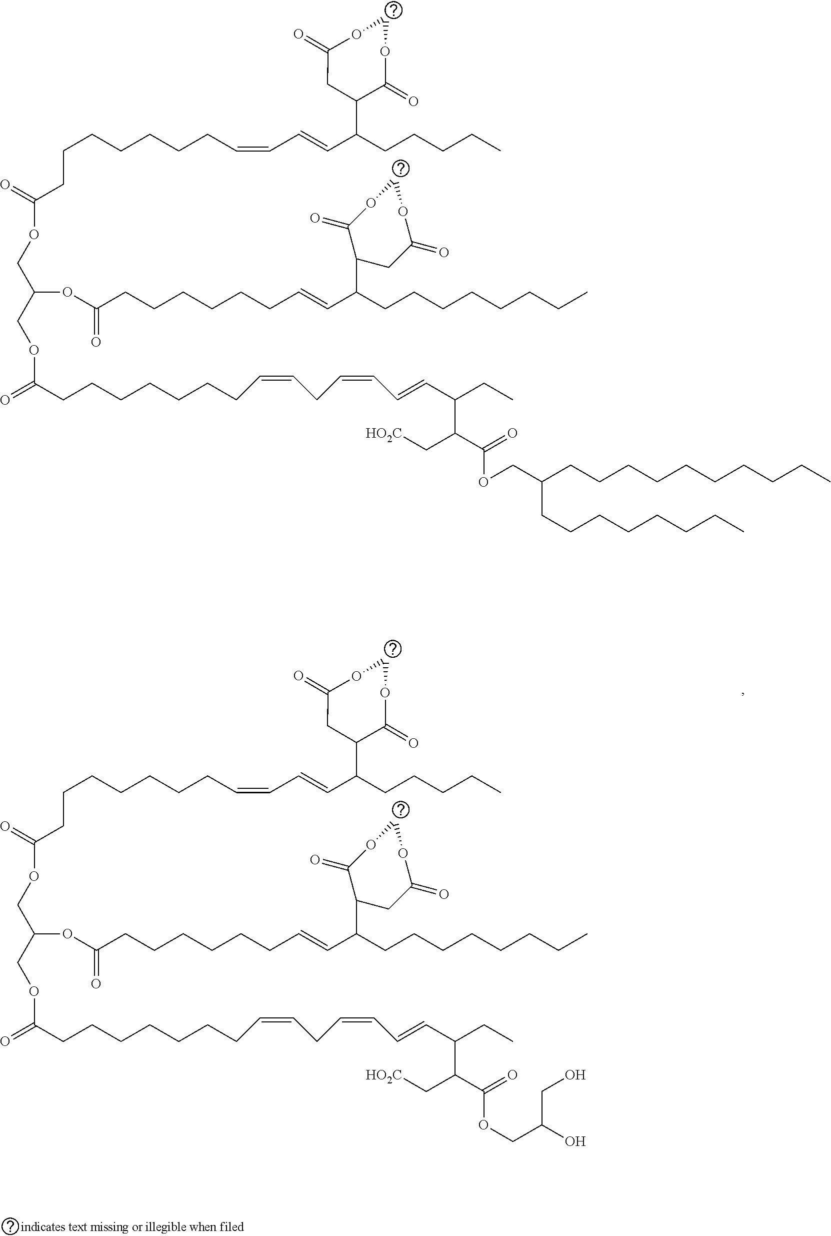

and combinations thereof. The reaction product above refers to fully or partially reacted succinic anhydride structures which may comprise succinic acid and any combination of alkali metals and alkaline earth metals, in any combination.

The oral care composition may be formulated as a toothpaste, tooth gel, subgingival gel, tooth powder, mouth-rinse, mouth wash, mousse, foam, denture product, mouth-spray, chewable tablet, dissolvable film, chewing gum, and mixtures thereof. The oral care composition may be an aqueous or non-aqueous based end-user composition. The oral care composition may have a pH in the range from about 4 to about 10.

The oral care composition may further comprise an orally acceptable excipient selected from the group consisting of thickening agents, desensitizing agents, whitening agents, tartar control agents, abrasives, binders, detergents, adhesion agents, foam modulators, pH modifying agents, mouth feel agents, sweeteners, flavorants, colorants, preservatives, humectants, fluoride containing salts and compounds, water, polymers, orally acceptable surfactants, and mixtures thereof. The oral care composition may further comprise an oral care active ingredient selected from the group consisting of an analgesic, an antibacterial, an antibiotic, a probiotic, an antioxidant, a peptide, an enzyme, a cooling agent, and mixtures thereof.

The present invention further provides a compound having the following structure:

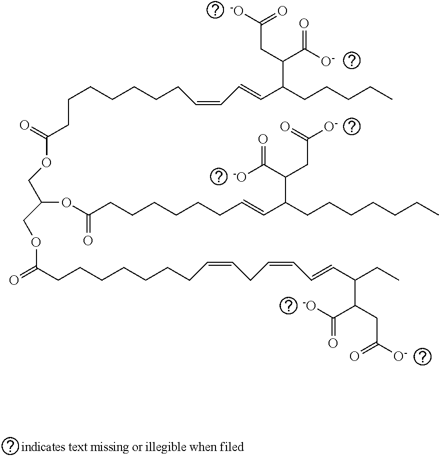

In one non-limiting embodiment, the metal base is selected from the group consisting of oxides, hydroxides, carbonates or bicarbonates of sodium or calcium or their combinations.

In one non-limiting embodiment, the hydrophobic moiety and the hydrophilic moiety is a hydrocarbyl alcohol, a hydrocarbyl amine, a silicon-based compound, or a combination thereof.

Hydrocarbyl alcohols are classified as primary, secondary and tertiary alcohols, based on the number of carbon atoms connected to the carbon atom that bears the hydroxyl group. Each classification of alcohol may have a general formula. For example,

the general formula for primary alcohols is

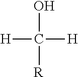

the general formula for secondary alcohols is

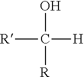

and
the general formula for tertiary alcohols is

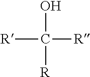

wherein R, R′ and R″ stand for hydrogen, different alkyl, alkylene, aryl, aralkyl, arylene, heteroatom groups.

In one non-limiting embodiment, the hydrophobic moiety hydrocarbyl alcohol and the hydrocarbyl amine contains C₂ to C₃₆ carbon atoms and is linear, branched, saturated, unsaturated, aliphatic, aromatic, monofunctional or multifunctional.

In one non-limiting embodiment, the hydrocarbyl alcohol is selected from the group consisting of monohydric alcohols, dihydric alcohols, polyhydric alcohols and combinations thereof.

In one non-limiting embodiment, the hydrocarbyl alcohol contains C₂ to C₃₆ carbon atoms and is linear, branched, saturated, unsaturated, aliphatic, aromatic, monofunctional or multifunctional.

In one non-limiting embodiment, the hydrocarbyl alcohol is selected from the group consisting of monohydric alcohols, dihydric alcohols, polyhydric alcohols and combinations thereof.

Hydrocarbyl amines are classified as primary, secondary and tertiary alcohols, based on the number of carbon atoms connected to the carbon atom that bears the hydroxyl group. Each classification of alcohol may have a general formula. For example,

the general formula for primary amine is

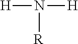

and
the general formula for secondary amine is

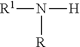

wherein R, R′ stand for hydrogen, different alkyl, alkylene, aryl, aralkyl, arylene, heteroatom groups.

In one non-limiting embodiment, the hydrocarbyl amine contains C₂ to C₃₆ carbon atoms and is linear, branched, saturated, unsaturated, aliphatic, aromatic, monofunctional or multifunctional.

In one non-limiting embodiment, the hydrocarbyl amine is selected from the group consisting of primary amines, secondary amines and combinations thereof.

In one non-limiting embodiment, the silicon-base compound is a

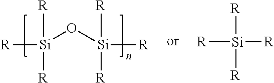

wherein R stands for different alkyl, alkylene, aryl, aralkyl, arylene, hetero groups functionalized with at least one or more alcohol, an amine or a combination thereof and n has the value of 1 to 10.

In one non-limiting embodiment, the silicon-base compound is a siloxane, or a silane functionalized with an alcohol, an amine or a combination thereof.

In one non-limiting embodiment, the silicon-based compound is a linear, branched, saturated, unsaturated, aliphatic, aromatic, monofunctional or multifunctional compound.

In one non-limiting embodiment, the hydrocarbyl alcohol is a hydrophobic alcohol selected from the group consisting of propanol, butanol, pentanol, heptanol, nonanol, decanol, dodecanol, phenol, ethylbenzyl alcohol, 2-ethyl-1-hexanol, 1-octanol, 2-octanol, 2-octyl-1-dodecyl alcohol, 2-tertradecanol, 2-hexadecanol, 3,7-dimethyl-1-octanol, 2-propyl-1-pentanol, 4-methyl-1-pentanol and mixtures thereof.

In one non-limiting embodiment, the hydrocarbyl alcohol is a hydrophilic alcohol selected from the group consisting of methanol, ethanol, propanol, isopropanol, butanol, ethylene glycol, propylene glycol, diethylene glycol, dipropylene glycol, dibutylene glycol, polyethylene glycol, methoxypolyethylene glycol, polypropylene glycol, hexylene glycol, glycerol, sorbitol, octanol, methyl ethyl pentanol, trimethyl pentanol, ethyl hexanol, methyl heptanol, nonanol, methyl octanol, ethyl heptanol, methyl ethyl hexanol, cyclohexanol, dimethyl heptanol, decanol, methyl nonanol, ethyl octanol, trimethyl heptanol, undecanol, methyl decanol, ethyl nonanol, dodecanol, tetradecanol, hexadecanol, octadecanol, benzyl alcohol, phenoxyethanol, neopentylglycol, trimethylol propane, methyldiethanol amine, erythritol, mannitol, xylitol, pentaerythritol, threitol, pentaerythritol, beta-cyclodextrin, L-Ribose, 2-deoxy-D-galactose and mixtures thereof.

In one non-limiting embodiment, the hydrocarbyl amine is hydrophobic amine selected from the group consisting of benzylamine, cyclohexylamine, hexylamine, methylhexylamine, phenethylamine, octylamine, oleylamine, decylamine, dodecylamine, octadecylamine, undecyl amine, pentadecyl amine, 2-methyl butyl amine, dimethyl amine and mixtures thereof.

In one non-limiting embodiment, the hydrocarbyl amine is hydrophilic amine selected from the group consisting of 2-methylpentane-1,5-diamine, diethanol amine, diisopropanolamine, serinol hydrochloride, 2-amino-2-ethyl-1, 3-propanediol, n-methyl-D-glucamine, D-galactosamine hydrochloride, d-glucosamine hydrochloride, D-mannosamine hydrochloride and mixtures thereof.

In one non-limiting embodiment, the silicon-based compound is a linear, branched, saturated, unsaturated, aliphatic, aromatic, monofunctional or multifunctional compound.

In one non-limiting embodiment, the silicon-base compound is a siloxane, or a silane functionalized with an alcohol, an amine or a combination thereof.

In one non-limiting embodiment, the silicon-based compound is a hydrophobic compound selected from the group consisting of aminopropylmethylsiloxane-dimethylsiloxane, N-ethylaminoisobutyl terminated polydimethylsiloxane, poly(1,1-dimethylsilazane) telomer, aminopropyl terminated polydimethylsiloxane, monoaminopropyl terminated polydimethylsiloxane, (tetramethylpiperidinyloxy)propylmethylsiloxane]-dimethylsiloxane copolymer, polydimethylsiloxane, carbinol (hydroxyl) terminated polydimethylsiloxane, monocarbinol terminated polydimethylsiloxane, monocarbinol terminated functional polydimethylsiloxane, [Bis(hydroxyethyl)amine] terminated polydimethylsiloxane, silanol terminated polydimethylsiloxane, silanol terminated polydiphenylsiloxane, dodecylmethylsiloxane-hydroxypolyalkyleneoxypropyl methylsiloxane and mixtures thereof.

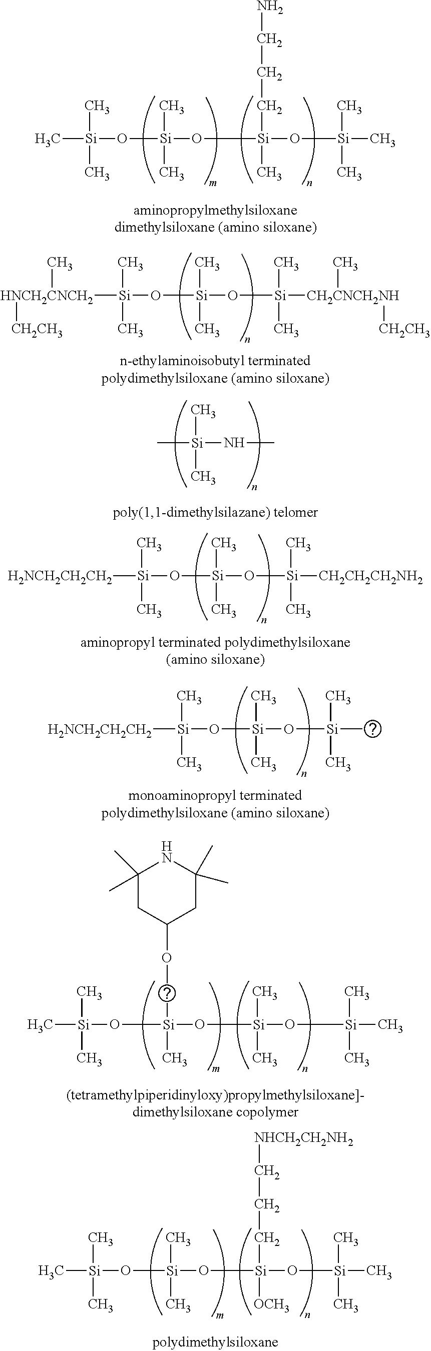

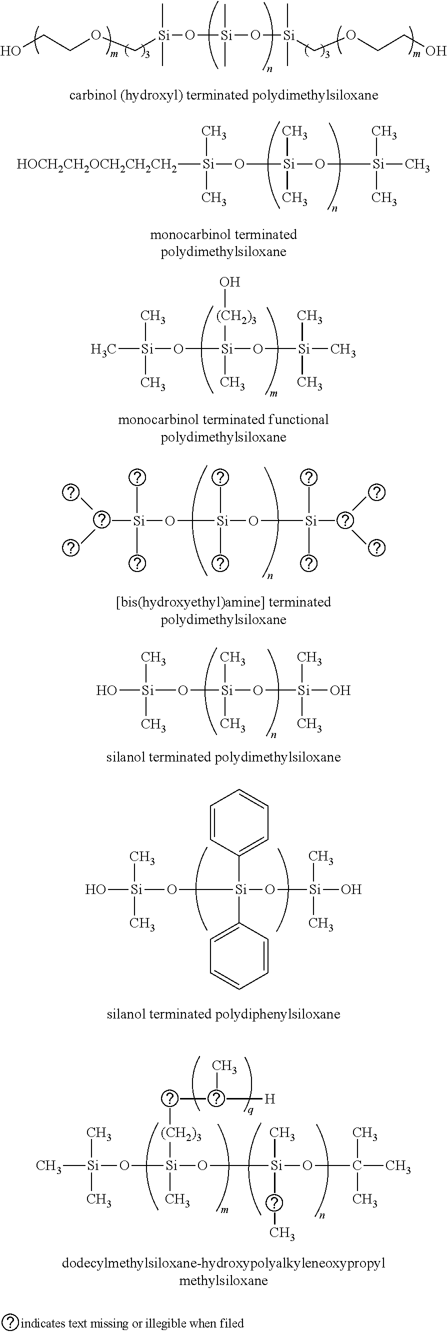

In one non-limiting embodiment, the silicon-based compound is hydrophilic compound selected from the group consisting of 3-aminopropylsilanetriol, N-(2-aminoethyl)-3-aminopropylsilanetriol and mixtures thereof.

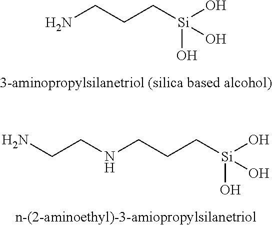 [SMILES: NCCC[Si](O)(O)O ; NCCNCCC[Si](O)(O)O]

In one non-limiting embodiment, the maleated natural oil is selected from the group consisting of a maleated avocado oil, a maleated coconut oil, a maleated corn oil, a maleated cottonseed oil, a maleated jojoba oil, a maleated linseed oil, a maleated nut oil, a maleated olive oil, a maleated palm oil, a maleated raisin oil, a maleated rapeseed oil, a maleated safflower oil, a maleated sesame oil, a maleated soybean oil, a maleated squash oil, a maleated sunflower oil, a maleated almond oil, a maleated canola oil, a maleated flaxseed oil, a maleated grapeseed oil, a maleated palm oil, a maleated palm kernel oil, a maleated peanut oil, and a maleated walnut oil thereof.

The reactions according to the application may be readily synthesized by procedures known by those skilled in the art, non-limiting examples of which include free radical solution polymerization, dispersion polymerization, emulsion polymerization, ionic chain polymerization, living polymerization, bulk polymerization, suspension polymerization or precipitation polymerization. Particularly, the polymerization is carried out by any one of the methods disclosed in “Principles of Polymerization” 4th edition, 2004, Wiley by George Odian and is referred and disclosed herein in its entirety and described in “Decomposition Rate of Organic Free Radical Polymerization” by K. W. Dixon (section II in Polymer Handbook, volume 1, 4th edition, Wiley-Interscience, 1999), which is herein incorporated in its entirety by reference.

The maleinization reaction in natural oil can occur under heating in three different ways. The first one is known as “Ene” reaction (reaction between an allylic hydrogen and an enophile-pericyclic reaction), obtaining a triglyceride structure with anhydride moieties (succinic anhydride). The second one is a radical addition, which consumes a double bond in the fatty acid, incorporating the succinic anhydride into the natural oil structure. The final reaction is also a radical addition that incorporates the maleic anhydride into the natural oil structure without consuming C═C bonds (fatty acid chain and maleic anhydride); this reaction occurs due to deprotonation of a hydrogen between two alkenes groups. Subsequently, the maleinized natural oil can be reacted with hydrophobic moiety and the hydrophilic moiety of a hydrocarbyl alcohol, a hydrocarbyl amine, a silicon-based compound forming a reticulated structure (see examples). This kind of reaction has been applied at the reaction occurred by heating 6-10 hours at 210° C.

Thus, the reaction of the non-limiting example above, for illustration purposes only, may be performed at elevated temperatures, such as a temperature between about 80° C. and about 45° C., alternatively between about 85° C. and about 90° C., or alternatively between about 90° C. and about 95° C. The reaction time may be between about 0.5 hours and about 10 hours. In one embodiment the reaction time is between about 1 hour and about 5 hours, and in another embodiment, between about 2 hours and 4 hours, and in another embodiment, between about 6 hours and 10 hours.

The desired pH of the oral care composition is an aqueous or non-aqueous based end-user composition is in the range of from about 4 to about 13, and in some embodiment, it is preferably between about 4 to about 10. The utility levels of the pH modifying agent may be present in an effective amount required to achieve the desired pH level.

During maleation, the mole ratio of natural oil to maleic anhydride in some embodiments is equal to 1, in other embodiments from 1 to 2, in other embodiments from 1 to 2.8 and in still other embodiments from 1 to 3.2 moles of maleic anhydride for each mole of natural oil.

During maleation, the mole ratio of maleated natural oil to one or more bases in some embodiments is equal to 1, in other embodiments from 1 to 2, in other embodiments from 1 to 2.8 and in still other embodiments from 1 to 3.2 moles of base for each mole of maleated natural oil.

The mole ratio of reaction of maleated natural oil and a hydrophobic moiety, a hydrophilic moiety or a combination thereof mixtures is equal to 1:1 to results a reaction product comprising one unreacted maleated functionality.

In one non-limiting embodiment, the composition is an oral care composition, a skin care composition, a hair care composition, an energy composition, a construction composition, a biocidal composition, a preservative composition, a nutraceutical composition, a food composition, an agricultural composition, a coating composition, a cosmetic composition, a homecare composition, an industrial and institutional composition, a textile composition, a laundry composition, a cleaning composition or a disinfecting composition.

According to another embodiment of the present application, it is contemplated to employ at least one orally acceptable excipients selected from the group consisting of thickening agents, desensitizing agents, whitening agents, tartar control agents, abrasives, binders, detergents, adhesion agents, foam modulators, pH modifying agents, mouth feel agents, sweeteners, flavorants, emulsifying agents, colorants, solubilizers, preservatives, humectants, fluoride containing salts and compounds, water, viscosity controlling agents, suspending agents, polymers, orally acceptable surfactants and mixtures thereof.

According to another embodiment of the present application, it is contemplated to employ at least one or more oral care active ingredients selected from the group consisting of an analgesic, an antibacterial, an anticalculus agents an antibiotic, a probiotic, an antioxidant, a peptide, an enzyme, a cooling agent, a preservative, a desensitizing agent, a dental remineralization agent, odor or breath freshening agents, warming agents, herbal agents, medicaments, vitamins, taste masking agents, pharmaceutical agent, a therapeutic agent, vitamin, a mineral, warming agent, a sensate, throat-soothing agent, spices, caffeine, drug and mixtures thereof.

Suitable antibacterial agents include, without limitation, copper (II) compounds such as copper (II) chloride, fluoride, sulfate and hydroxide, zinc ion sources such as zinc acetate, zinc citrate, zinc gluconate, zinc glycinate, zinc oxide, zinc sulfate and sodium zinc citrate, phthalic acid and salts thereof such as magnesium monopotassium phthalate, hexetidine, octenidine, sanguinarine, benzalkonium chloride, domiphen bromide, alkylpyridinium chlorides such as cetylpyridinium chloride (CPC) (including combinations of CPC with zinc and/or enzymes), tetradecylpyridinium chloride and N-tetradecyl-4-ethylpyridinium chloride, iodine, sulfonamides, bisbiguanides such as alexidine, chlorhexidine and chlorhexidine digluconate, piperidino derivatives such as delmopinol and octapinol, magnolia extract, grapeseed extract, raspberry ketone, menthol, geraniol, citral, cucalyptol, eugenol, stannous fluoride, magnolia bark extracts, Chinese traditional medicines, thymol, 4-isopropyl m-cresol (IPMP, o-cymen 5-ol) antibiotics such as amoxicillin, tetracycline, doxycycline, minocycline, metronidazole, neomycin, kanamycin and clindamycin, and the like. A further illustrative list of useful antibacterial agents is provided in U.S. Pat. No. 5,776,435 to Gaffar et al.

Suitable flavorants include those flavors known to the skilled artisan, such as natural and artificial flavors. These flavorings chosen from synthetic flavor oils and flavoring aromatics and/or oils, oleoresins and extracts derived from plants, leaves, flowers, fruits, and so forth, and combinations thereof. Nonlimiting representative flavor oils include spearmint oil, cinnamon oil, oil of wintergreen (methyl salicylate), peppermint oil, clove oil, bay oil, anise oil, eucalyptus oil, thyme oil, cedar leaf oil, oil of nutmeg, allspice, oil of sage, mace, oil of bitter almonds, and cassia oil. Also useful flavorings are artificial, natural and synthetic fruit flavors such as vanilla, and citrus oils including lemon, orange, lime, grapefruit, and fruit essences including apple, pear, peach, grape, blueberry, strawberry, raspberry, cherry, plum, pineapple, apricot and so forth. These flavoring agents may be used in liquid or solid form and may be used individually or in admixture. Commonly used flavors include mints such as peppermint, menthol, spearmint, artificial vanilla, cinnamon derivatives, and various fruit flavors, whether employed individually or in admixture. Flavors may also provide breath freshening properties, particularly the mint flavors when used in combination with the cooling agents, described herein below.

Non-limiting examples of suitable animal and vegetable oils include: sunflower oil, corn oil, soy oil, avocado oil, jojoba oil, squash oil, raisin seed oil, sesame seed oil, walnut oil, fish oil, glycerol tricaprocaprylate, purcellin oil, liquid jojoba, and blends thereof. Also suitable are natural oils such as oils of eucalyptus, lavender, vetiver, litsea cubeba, lemon, sandalwood, rosemary, chamomile, savory, nutmeg, cinnamon, hyssop, caraway, orange, geranium, cade, bergamot, and blends thereof. There are five commonly available vegetable oils which contain greater than 50% linoleic acid groups (as their glycerol esters). These are, in decreasing order of linoleic content: Safflower Oil (75%); Sunflower Oil (67.8%); Corn Oil (56%); Cottonseed Oil (55%) and Soybean Oil (52%).

Other useful flavorings include aldehydes, esters and ketones such as cinnamyl acetate, cinnamaldehyde, citral diethylacetal, dihydrocarvyl acetate, eugenyl formate, raspberry ketone p-methylamisol, and so forth may be used. Generally, any flavoring or food additive such as those described in Chemicals Used in Food Processing, publication 1274, pages 63-258, by the National Academy of Sciences, may be used. This publication is incorporated herein by reference. This may include natural as well as synthetic flavors.

A variety of other nutritional supplements may also be included, such as vitamin or mineral as mentioned above. For example, vitamin A, vitamin C, vitamin D, vitamin E, vitamin K, vitamin B6, vitamin B12, thiamine, riboflavin, biotin, folic acid, niacin, pantothenic acid, sodium, potassium, calcium, magnesium, iron, copper, zinc, selenium, manganese, choline, chromium, molybdenum, cobalt and combinations thereof, may be used.

In another embodiment, whitening agents such as blue covarine, blue colors and dyes, polyphosphates, phytic acid and its' salts, complexed bleaching or bleaching agents. Suitable bleaching or whitening agents include peroxide compounds. Peroxides are believed to whiten teeth by releasing hydroxyl radicals capable of breaking down the plaque-stain complex into a form that can be flushed away or removed by abrasives. Useful peroxides should contain an O—O bond, which can break down to provide at least one active species. Examples of preferred peroxide compounds are inorganic peroxides, such as hydrogen peroxide, calcium peroxide, strontium peroxide, zinc peroxide or magnesium peroxide, and organic peroxides including, but not limited to, carbamide peroxide, PVP-hydrogen peroxide complexes and sodium percarbonate.

Moreover, in some embodiments a film-forming polymer can be included in the compositions of the present invention. For example, the film-forming polymer may be a synthetic anionic polymeric polycarboxylate (SAPP), such a PVM/MA copolymer (Gantrez S-97, Ashland Inc.).

Such polymers are described in U.S. Pat. Nos. 5,334,375 and 5,505,933, which are incorporated by reference herein in their entirety. SAPP's have previously been described as useful for dentin sensitivity reduction. Moreover, SAPP's have previously been described as antibacterial-enhancing agents, which enhance delivery of an antibacterial agent to oral surfaces, and which enhance the retention of the antibacterial agent on oral surfaces. It is well within the contemplation of the present invention that film-forming polymers, such as PVM/MA copolymer, may be employed in the compositions of the present invention as a means of reducing stain formation.

In another embodiment, the sweetening agents which can include but not limited to sucrose, glucose, saccharin, dextrose, levulose, lactose, mannitol, sorbitol, fructose, maltose, xylitol, saccharin salts, thaumatin, aspartame, D-tryptophan, dihydrochalcones, acesulfame, sucralose and cyclamate salts, especially sodium cyclamate and sodium saccharin, and mixtures thereof.

In another embodiment, the present application further comprises suitable abrasives include but not limited to silica abrasives, such as standard cleaning silicas, high cleaning silicas or any other suitable abrasive silicas. Additional examples of abrasives that can be used in addition to or in place of the silica abrasives include, for example, a calcium phosphate abrasive, e.g., tricalcium phosphate (Ca3(PO4)2), hydroxyapatite (Ca10(PO4)6(OH)2), or dicalcium phosphate dihydrate CaHPO4.2H2O or calcium pyrophosphate; calcium carbonate abrasive; or abrasives such as sodium metaphosphate, potassium metaphosphate, aluminum silicate, calcined alumina, bentonite or other siliceous materials, or combinations thereof. The silica component of the present silica substrate is an amorphous precipitated silica. Precipitated silicas include the following products available from the J. M. Huber Corporation, Edison, N.J.: Zeodent® 103, Zeodent® 113, Zeodent® 114, Zeodent® 115, Zeodent® 118, Zeodent® 119, Zeodent® 165, and Zeodent® 9175.

In another embodiment, the present application anionic surfactants include water-soluble salts of alkyl sulfates having from 8 to 20 carbon atoms in the alkyl radical (e.g., sodium alkyl sulfate) and the water-soluble salts of sulfonated monoglycerides of fatty acids having from 8 to 20 carbon atoms, such as sodium lauryl sulfate and sodium coconut monoglyceride sulfonate. Other suitable anionic surfactants are sarcosinates, such as sodium lauroyl sarcosinate, taurates, sodium lauryl sulfoacetate, sodium lauroyl isethionate, sodium lauryl carboxylate, and sodium dodecyl benzenesulfonate, and mixtures thereof.

Another surfactant is selected from the group consisting of sarcosinate surfactants, isethionate surfactants and taurate surfactants. Surfactants are alkali metal or ammonium salts of these surfactants, sodium and potassium salts of lauroyl sarcosinate, myristoyl sarcosinate, palmitoyl sarcosinate, stearyl sarcosinate, and oleoyl sarcosinate.

Cationic surfactants are derivatives of aliphatic quaternary ammonium compounds having one long alkyl chain containing from about 8 to 18 carbon atoms such as lauryl trimethylammonium chloride; cetyl pyridinium chloride; cetyl trimethylammonium bromide; di-isobutylphenoxyethyl-dimethylbenzylammonium chloride; coconut alkyltrimethylammonium nitrite; cetyl pyridinium fluoride; etc.

Examples of suitable nonionic surfactants include the Pluronics, polyethylene oxide condensates of alkyl phenols, products derived from the condensation of ethylene oxide with the reaction product of propylene oxide and ethylene diamine, ethylene oxide condensates of aliphatic alcohols, long chain tertiary amine oxides, long chain tertiary phosphine oxides, long chain dialkyl sulfoxides and mixtures of such materials.

Zwitterionic are derivatives of aliphatic quaternary ammonium, phosphonium, and sulfonium compounds, in which the aliphatic radicals can be straight chain or branched, and wherein one of the aliphatic substituents contains from about 8 to 18 carbon atoms and one contains an anionic water-solubilizing group, e.g., carboxy, sulfonate, sulfate, phosphate or phosphonate.

In another embodiment, the present application, thickening agents includes water-soluble cellulose ethers such as sodium carboxymethylcellulose, sodium carboxymethyl hydroxyethyl cellulose and, hydroxyethyl cellulose, PVP generally refers to a polymer containing vinylpyrrolidone (also referred to as N-vinylpyrrolidone, N-vinyl-2-pyrrolidone and N-vinyl-2-pyrrolidinone) as a monomeric unit. Other suitable thickening agents include carboxyvinyl polymers, carrageenan, laponite and other natural gums such as gum karaya, xanthan gum, guar gum, gum arabic, and gum tragacanth. Colloidal magnesium aluminum silicate or finely divided silica can be used as part of the thickening agent to further improve texture. A class of thickening or gelling agents includes a class of homopolymers of acrylic acid cross-linked with an alkyl ether of pentaerythritol or an alkyl ether of sucrose.

In another embodiment, the present application, anti-microbial agents can include but not limited to halogenated diphenyl ether, 2,4,4′-trichloro-2′-hydroxy-diphenyl ether, 2,2′-dihydroxy-5,5′-dibromo-diphenyl ether, 2,2′-methylenebis-4 (4-chloro-6-bromo-phenol), halogenated salicylanilides and halogenated cabanilides, stannous chloride, zinc lactate, zinc citrate, zinc oxide.

As used herein “fluoride-providing compound” can include but not limited to sodium fluoride, potassium fluoride, amine fluoride, ammonium fluoride, lead fluoride, manganese fluoride, a copper fluoride such as cuprous fluoride, zinc fluoride, barium fluoride, sodium fluorosilicate, ammonium fluorosilicate, sodium fluorozirconate, sodium monofluorophosphate, potassium fluorozirconates, sodium monofluorophosphate, aluminum mono- and di-fluorophosphate, and fluorinated sodium calcium pyrophosphate, a tin fluoride such as stannous fluoride, stannic fluoride or stannous chlorofluoride, and sodium hexafluorostannate.

According to another embodiment of the present application, improve oral health comprising applying an effective amount of the oral composition to an oral cavity surface, the method is effective (i) to reduce or inhibit formation of dental caries, (ii) for preventing the demineralization of the teeth, (iii) to promote remineralization of the teeth, (iv) to reduce or inhibit hypersensitivity of the teeth, (v) to treat gingivitis, erosion, cavities, calculus, inflammation, staining and plaque accumulation, and (vi) to reduce biofilm formation, (vii) enhance antibacterial activity and (viii) improve flavor retention.

According to another embodiment of the present application, oral health comprising an effective amount of the oral composition of claim 1, wherein the method is effective at delivering and retaining therapeutic agents such as; (i) antibacterial, (ii) anti-caries, (iii) anti-gingivitis, (iv) anti-inflammation, (v) anti-bio-film, (vi) anti-staining and (vii) anti-erosion.

In some embodiments, the suitable range of a reaction product of a maleated natural oil and one or more bases can be varied from about 0.1 wt. % to about 1 wt. %, or from about 1 wt. % to about 5 wt. %, or from about 5 wt. % to about 10 wt. % or 10 wt. % to about 15 wt. %; or from about 15 wt. % to about 20 wt. %; or from about 20 wt. % to about 25 wt. %; or from about 25 wt. % to about 30 wt. %; or from about 30 wt. % to about 35 wt. %; or from about 35 wt. % to about 40 wt. %; or from about 40 wt. % to about 45 wt. %; or from about 45 wt. % to about 50 wt. %; or from about 50 wt. % to about 55 wt. %; or from about 55 wt. % to about 60 wt. %; or from about 60 wt. % to about 65 wt. %; or from about 65 wt. % to about 70 wt. %; or from about 70 wt. % to about 75 wt. %; or from about 75 wt. % to about 80 wt. %; or from about 80 wt. % to about 85 wt. %; or from about 85 wt. % to about 90 wt. % or from about 90 wt. % to about 95 wt. %, or from about 95 wt. % to about 99.9 wt. % based on the total weight of the oral care antimicrobial care composition.

In some embodiments, the suitable range of orally acceptable excipients for the present application can be varied from about 0.1 wt. % to about 1 wt. %; or from about 1 wt. % to about 2.5 wt. %; or from about 2.5 wt. % to about 5 wt. %; or from about 5 wt. % to about 10 wt. %; or 10 wt. % to about 15 wt. %; or from about 15 wt. % to about 20 wt. %; or from about 20 wt. % to about 25 wt. %; or from about 25 wt. % to about 30 wt. %; or from about 30 wt. % to about 35 wt. %; or from about 35 wt. % to about 40 wt. %; or from about 40 wt. % to about 45 wt. %; or from about 45 wt. % to about 50 wt. %; or from about 50 wt. % to about 55 wt. %; or from about 55 wt. % to about 60 wt. %; or from about 60 wt. % to about 65 wt. %; or from about 65 wt. % to about 70 wt. %; or from about 70 wt. % to about 75 wt. %; or from about 75 wt. % to about 80 wt. %; or from about 80 wt. % to about 85 wt. %; or from about 85 wt. % to about 90 wt. % or from about 90 wt. % to about 95 wt. %, or from about 95 wt. % to about 99.9 wt. % based on the total weight of the oral care antimicrobial

In some embodiments, the suitable range of oral care active ingredient for the present application can be varied from about 0.1 wt. % to about 1 wt. %; or from about 1 wt. % to about 2.5 wt. %; or from about 2.5 wt. % to about 5 wt. %; or from about 5 wt. % to about 10 wt. %; or 10 wt. % to about 15 wt. %; or from about 15 wt. % to about 20 wt. %; or from about 20 wt. % to about 25 wt. %; or from about 25 wt. % to about 30 wt. %; or from about 30 wt. % to about 35 wt. %; or from about 35 wt. % to about 40 wt. %; or from about 40 wt. % to about 45 wt. %; or from about 45 wt. % to about 50 wt. % based on the total weight of the oral care antimicrobial composition.

As per another embodiment of the present application, the oral care composition is formulated as a toothpaste, tooth gel, subgingival gel, tooth powder, mouth-rinse, mouth wash, mousse, foam, denture product, mouth-spray, chewable tablet, dissolvable film or chewing gum.

The reactions and compositions according to the application may be analyzed by known techniques. Especially preferred are the techniques of 13C nuclear magnetic resonance (NMR) spectroscopy, gas chromatography (GC), Infra-red (IR), Liquid Chromatography (LC) and gel permeation chromatography (GPC) in order to decipher identity, residual monomer concentrations, molecular weight, and molecular weight distribution.

Further, certain aspects of the present application are illustrated in detail by way of the following examples. The examples are given herein for illustration of the application and are not intended to be limiting thereof.

### EXAMPLES

### Example A: Grafting of Maleic Anhydride onto Natural Oils

### Example A 1: Grafting of Maleic Anhydride onto Soybean Oil

In a 1-Liter, 4 neck kettle equipped with a thermocouple, a condenser, a nitrogen sparge adapter, and a mechanical stirrer, 600 g of yellow color Soybean oil and 204 g (3 mole eq. based on SBO) of Maleic anhydride were charged. The mixture was sparged with nitrogen for 15 min at RT only. The mixture was slowly heated from room temperature to 210° C. and was held isothermally at 210° C. for about 6-8 hours. Completion of the reaction was indicated by NMR and LC<0.05% residual maleic anhydride.

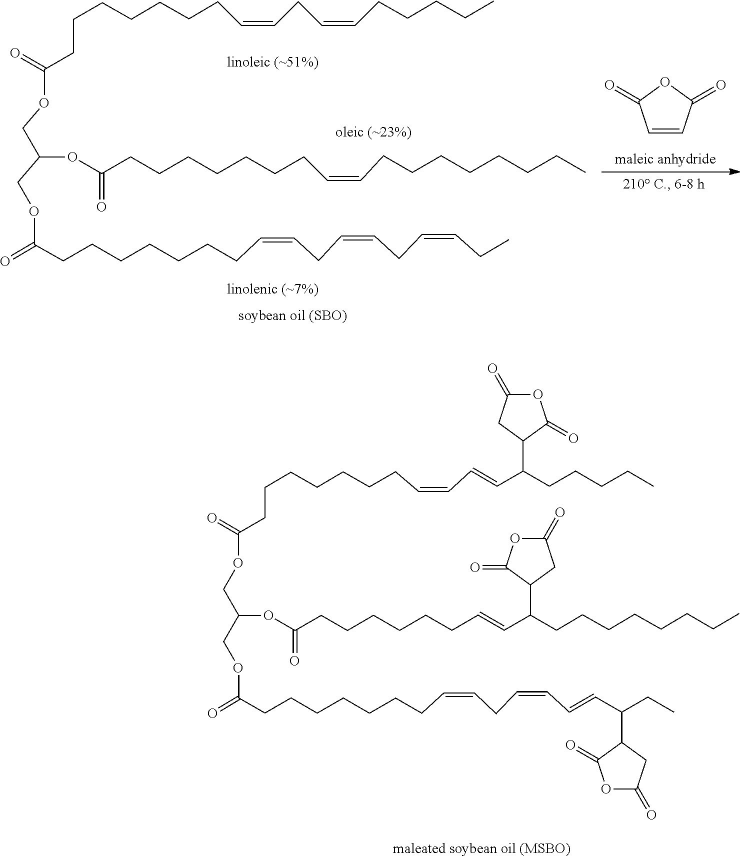 [SMILES: CC/C=C\C/C=C\C/C=C\CCCCCCCC(=O)OCC(COC(=O)CCCCCCC/C=C\C/C=C\CCCCC)OC(=O)CCCCCCC/C=C\CCCCCCCC ; O=C1C=CC(=O)O1 ; CCCCCCCCC(/C=C/CCCCCCC(=O)OC(COC(=O)CCCCCCC/C=C\C=C\C(CCCCC)C1CC(=O)OC1=O)COC(=O)CCCCCCC/C=C\C/C=C\C=C\C(CC)C1CC(=O)OC1=O)C1CC(=O)OC1=O]

### Example A 2: Grafting of Maleic Anhydride onto Palm Oil

In a 1-Liter, 4 neck kettle equipped with a thermocouple, a condenser, a nitrogen sparge adapter, and a mechanical stirrer, 100 g of Palm oil and 23 g (2 mole eq. based on palm oil) of Maleic anhydride was charged. The mixture was sparged with nitrogen for 15 min at RT only. The mixture was slowly heated from room temperature to 210° C. and held isothermally at 210° C. for about 8-10 hours. The one pot reaction without catalyst and solvent yields >96% amber color viscous product which was characterized by NMR and LC<1% residual maleic anhydride.

### Example A 3: Grafting of Maleic Anhydride onto Canola Oil

In a 1-Liter, 4 neck kettle equipped with a thermocouple, a condenser, a nitrogen sparge adapter, and a mechanical stirrer, 100 g of Canola oil and 22.2 g (2 mole eq. based on canola oil) of Maleic anhydride was charged. The mixture was sparged with nitrogen for 15 min at RT only. The mixture was slowly heated from room temperature to 210° C. and held isothermally at 210° C. for about 8-10 hours. The one pot reaction without catalyst and solvent yields >96% amber color viscous product which was characterized by NMR and LC<1% residual maleic anhydride.

### Example A 4: Grafting of Maleic Anhydride onto Sunflower Oil

In a 1-Liter, 4 neck kettle equipped with a thermocouple, a condenser, a nitrogen sparge adapter, and a mechanical stirrer, 100 g of Sunflower oil and 22.4 g (2 mole eq. based on sunflower oil) of Maleic anhydride was charged. The mixture was sparged with nitrogen for 15 min at RT only. The mixture was slowly heated from room temperature to 210° C. and held isothermally at 210° C. for about 8-10 hours. The one pot reaction without catalyst and solvent yields >96% amber color viscous product which was characterized by NMR and LC<1% residual maleic anhydride.

### Example A 5: Grafting of Maleic Anhydride onto Castor Oil

In a 1-Liter, 4 neck kettle equipped with a thermocouple, a condenser, a nitrogen sparge adapter, and a mechanical stirrer, 100 g of yellow color Castor oil and 21 g (2 mole eq. based on castor oil) of Maleic anhydride was charged. The mixture was sparged with nitrogen for 15 min at RT only. The mixture was slowly heated from room temperature to 210° C. and held isothermally at 210° C. for about 8-10 hours. The one pot reaction without catalyst and solvent yields >96% amber color viscous product which was characterized by NMR and LC<1% residual maleic anhydride.

### Example B: Salts of Maleated Natural Oils

### Example B 1: Sodium Salt of Maleated Soybean Oil from Aqueous Sodium Hydroxide

In a 1-L, 4 neck kettle equipped with a thermocouple, a condenser, a nitrogen purge adapter, and a mechanical stirrer, 100 g of amber color viscous product from maleination reaction A1 was heated to 90° C. and added a mixture of 20.20 g (3 mole eq.) of 50% aqueous sodium hydroxide and 70 g (46 mole eq.) water over 1 hour controlling foaming and held for 3 hours. The reaction yields >96% flowable amber color 70% active water-soluble product which was characterized by IR, NMR and LC<0.05% residual maleic anhydride.

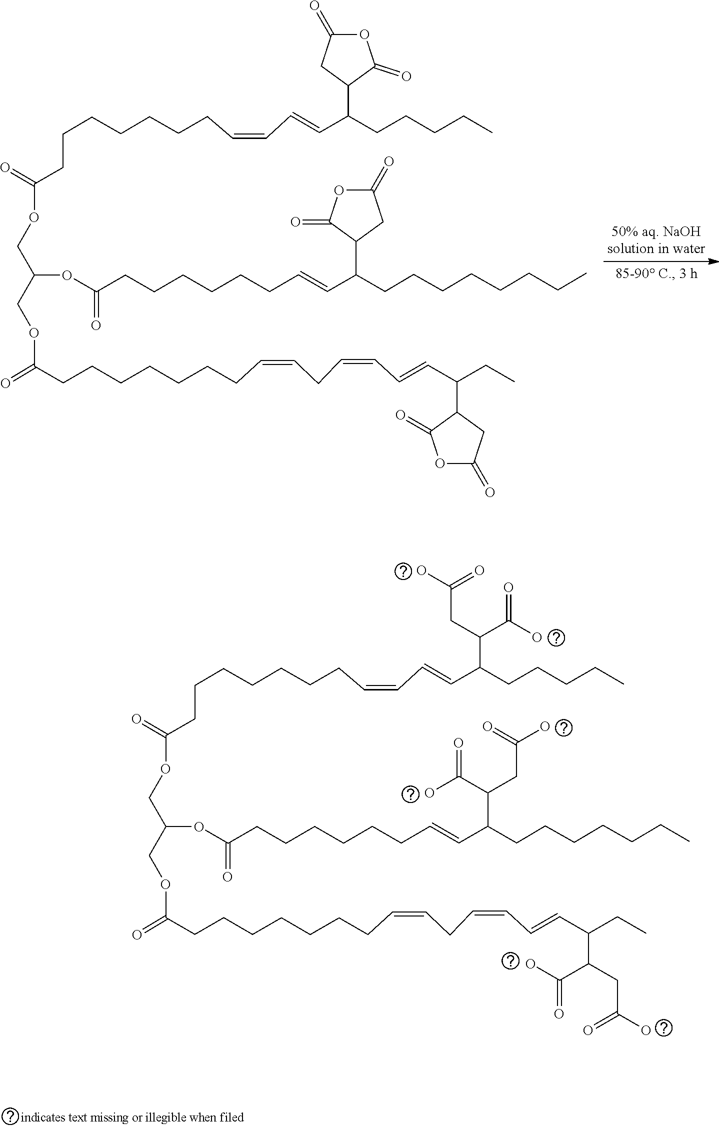 [SMILES: CCCCCCCCC(/C=C/CCCCCCC(=O)OC(COC(=O)CCCCCCC/C=C\C=C\C(CCCCC)C1CC(=O)OC1=O)COC(=O)CCCCCCC/C=C\C/C=C\C=C\C(CC)C1CC(=O)OC1=O)C1CC(=O)OC1=O ; CCCCCCCC(/C=C/CCCCCCC(=O)OC(COC(=O)CCCCCCC/C=C\C=C\C(CCCCC)C(CC(=O)O)C(=O)O)COC(=O)CCCCCCC/C=C\C/C=C\C=C\C(CC)C(CC(=O)O)C(=O)O)C(CC(=O)O)C(=O)O]

### Example B 2: Sodium Salt of Maleated Palm Oil from Aqueous Sodium Hydroxide

In a 1-Liter, 4 neck kettle equipped with a thermocouple, a condenser, a nitrogen purge adapter, and a mechanical stirrer, 123 g of amber color viscous product from maleination reaction A 2 was heated to 90° C. and add a mixture of 18.04 g (2 mole eq.) of 50% aqueous sodium hydroxide and 45 g (22 mole eq.) water over 1 hour controlling foaming and held for 3 hours. The reaction yields >96% flowable amber color 70% active water-soluble product which was characterized by IR, NMR and LC<0.05% residual maleic anhydride.

### Example B 3: Sodium Salt of Maleated Canola Oil from Aqueous Sodium Hydroxide

In a 1-Liter, 4 neck kettle equipped with a thermocouple, a condenser, a nitrogen purge adapter, and a mechanical stirrer, 123 g of amber color viscous product from maleination reaction A 3 was heated to 90° C. and add a mixture of 18.25 g (2 mole eq.) of 50% aqueous sodium hydroxide and 45 g (22 mole eq.) water over 1 hour controlling foaming and held for 3 hours. The reaction yields >96% flowable amber color 70% active water-soluble product which was characterized by IR, NMR and LC<0.05% residual maleic anhydride.

### Example B 4: Sodium Salt of Maleated Sunflower Oil from Aqueous Sodium Hydroxide

In a 1-Liter, 4 neck kettle equipped with a thermocouple, a condenser, a nitrogen purge adapter, and a mechanical stirrer, 123 g of amber color viscous product from maleination reaction A 4 heated to 90° C. and add a mixture of 18.26 g (2 mole eq.) of 50% aqueous sodium hydroxide and 45 g (22 mole eq.) water over 1 hour controlling foaming and was held for 3 hours. The reaction yields >96% flowable amber color 70% active water-soluble product which was characterized by IR, NMR and LC<0.05% residual maleic anhydride.

### Example B 5: Sodium Salt of Maleated Castor Oil from Aqueous Sodium Hydroxide

In a 1-Liter, 4 neck kettle equipped with a thermocouple, a condenser, a nitrogen purge adapter, and a mechanical stirrer, 123 g of amber color viscous product from maleination reaction A 5 was heated to 90° C. and add a mixture of 17.15 g (2 mole eq.) of 50% aqueous sodium hydroxide and 45 g (23 mole eq.) water over 1 hour controlling foaming and was held for 3 hours. The reaction yields >96% flowable amber color 70% active water-soluble product which was characterized by IR, NMR and LC<0.05% residual maleic anhydride.

### Example C 1: Sodium Salt of Maleated Soybean Oil from Sodium Carbonate

In a 1-Litre, 4 neck kettle equipped with a thermocouple, a condenser, a nitrogen purge adapter, and a mechanical stirrer, 100 g of amber color viscous product from maleination reaction A1 was heated to 90° C. and added a mixture of 27.86 g (3 mole eq.) of sodium carbonate dissolved in 85 g (55 mole eq.) water over 3 hours controlling the foam formation. The reaction was held at 90° C. for 3 hours. The reaction yields >50% flowable light yellow color 56% active water-soluble product which was characterized by IR, and LC<0.05% residual maleic anhydride.

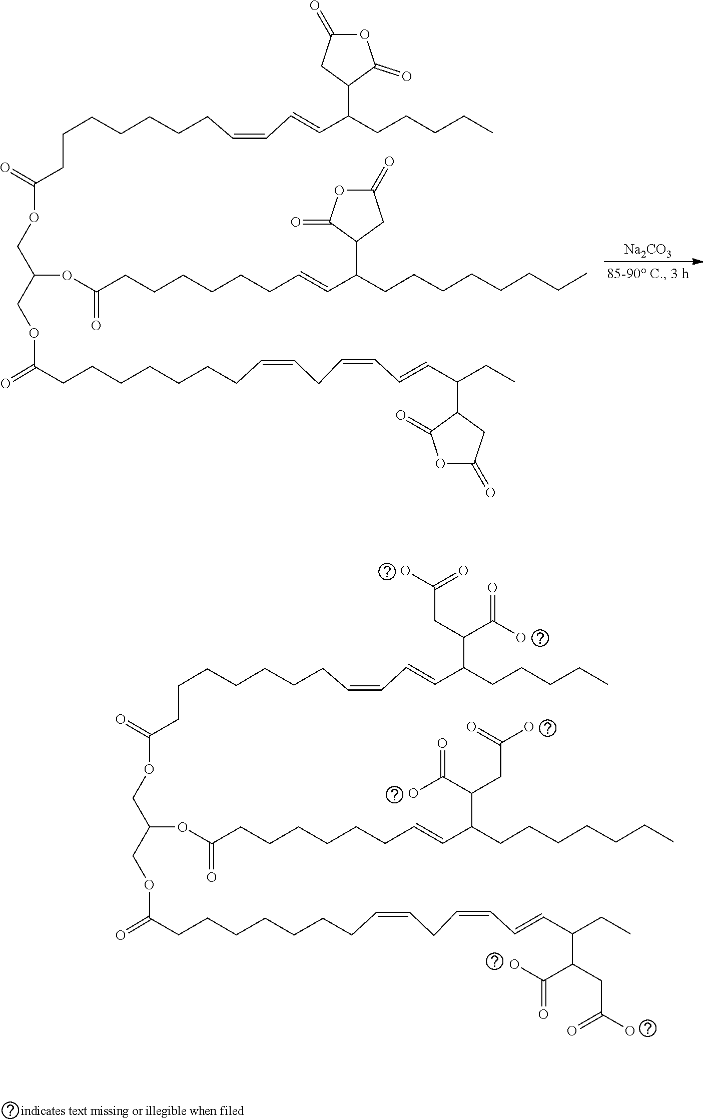

### Example D: Sodium Calcium Salt of Maleated Natural Oil

### Example D 1: Sodium Calcium Salt of Maleated Soybean Oil from Calcium Carbonate and Sodium Carbonate (7:3 Ratio)

In a 1-Liter, 4 neck kettle equipped with a thermocouple, a condenser, a nitrogen purge adapter, and a mechanical stirrer, 100 g of amber color viscous product from maleination reaction A1 was heated to 90° C. and added a mixture of 8.36 g (0.9 mole eq.) of sodium carbonate and 17.8 g (2.1 mole eq.) of calcium carbonate dissolved in 85 g (55 mole eq.) water over 3 hours controlling the foam formation. The reaction was held at 90° C. or 3 hours. The reaction yields >90% flowable light yellow color 60% active water-soluble product which was characterized by IR, and LC<0.05% residual maleic anhydride.

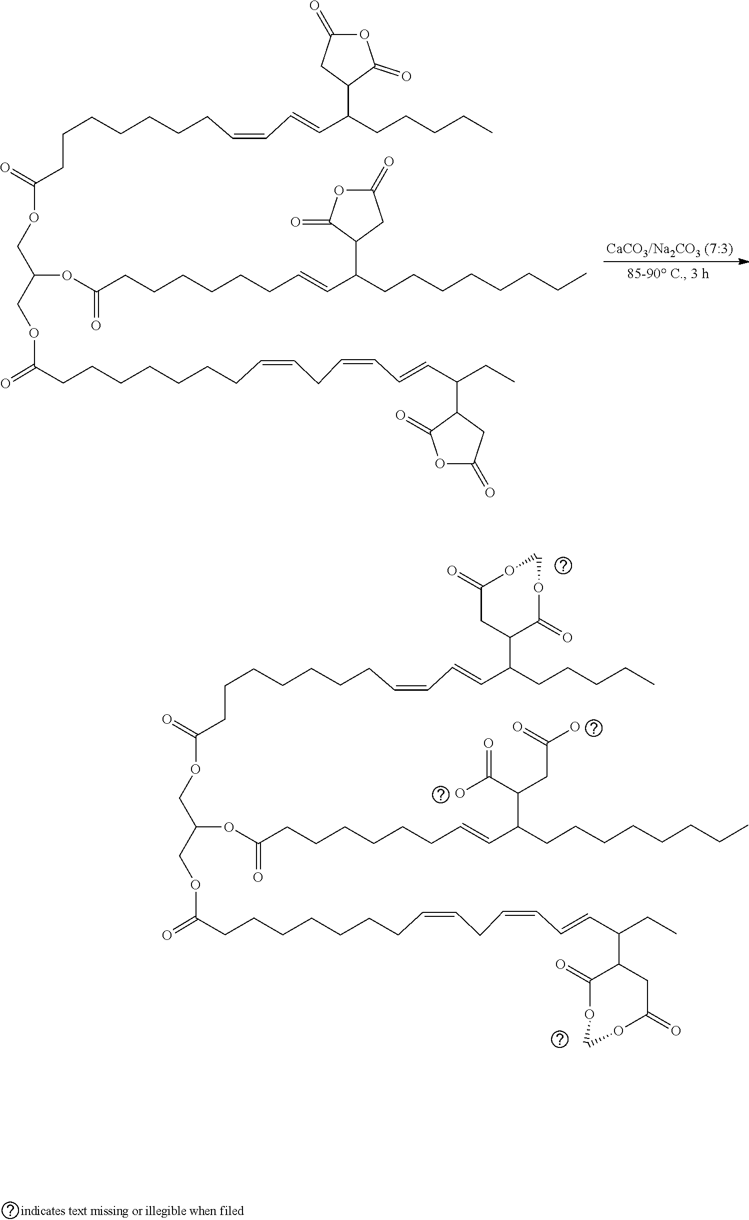

### Example D 2: Sodium Calcium Salt of Maleated Palm Oil from Calcium Carbonate and Sodium Carbonate (7:3 Ratio)

In a 1-Liter, 4 neck kettle equipped with a thermocouple, a condenser, a nitrogen purge adapter, and a mechanical stirrer, 122 g of amber color viscous product from maleination reaction A 2 was heated to 90° C. and added a mixture of 11.55 g (0.9 mole eq.) of sodium carbonate and 24.6 g (2.1 mole eq.) of calcium carbonate dissolved in 122 g (58 mole eq.) water over 3 hours controlling the foam formation. The reaction was held at 90° C. or 3 hours. The reaction yields >90% flowable light yellow color 60% active water-soluble product which was characterized by IR, and LC<0.05% residual maleic anhydride.

### Example D 3: Sodium Calcium Salt of Maleated Canola Oil from Calcium Carbonate and Sodium Carbonate (7:3 Ratio)

In a 1-Liter, 4 neck kettle equipped with a thermocouple, a condenser, a nitrogen purge adapter, and a mechanical stirrer, 122 g of amber color viscous product from maleination reaction A 3 was heated to 90° C. and added a mixture of 11.3 g (0.9 mole eq.) of sodium carbonate and 24 g (2.1 mole eq.) of calcium carbonate dissolved in 122 g (60 mole eq.) water over 3 hours controlling the foam formation. The reaction was held at 90° C. or 3 hours. The reaction yields >90% flowable light yellow color 60% active water-soluble product which was characterized by IR, and LC<0.05% residual maleic anhydride.

### Example D 4: Sodium Calcium Salt of Maleated Sunflower Oil from Calcium Carbonate and Sodium Carbonate (7:3 Ratio)

In a 1-Liter, 4 neck kettle equipped with a thermocouple, a condenser, a nitrogen purge adapter, and a mechanical stirrer, 122 g of amber color viscous product from maleination reaction A 4 was heated to 90° C. and added a mixture of 11.55 g (0.9 mole eq.) of sodium carbonate and 24.6 g (2.1 mole eq.) of calcium carbonate dissolved in 122 g (58 mole eq.) water over 3 hours controlling the foam formation. The reaction was held at 90° C. or 3 hours. The reaction yields >90% flowable light yellow color 60% active water-soluble product which was characterized by IR, and LC<0.05% residual maleic anhydride.

### Example D 5: Sodium Calcium Salt of Maleated Castor Oil from Calcium Carbonate and Sodium Carbonate (7:3 Ratio)

In a 1-Liter, 4 neck kettle equipped with a thermocouple, a condenser, a nitrogen purge adapter, and a mechanical stirrer, 122 g of amber color viscous product from maleination reaction A 5 was heated to 90° C. and added a mixture of 10.6 g (0.9 mole eq.) of sodium carbonate and 22.5 g (2.1 mole eq.) of calcium carbonate dissolved in 122 g (63 mole eq.) water over 3 hours controlling the foam formation. The reaction was held at 90° C. or 3 hours. The reaction yields >90% flowable light yellow color 60% active water-soluble product which was characterized by IR, and LC<0.05% residual maleic anhydride.

### Example E 1: Calcium Salt of Maleated Soybean Oil from Calcium Carbonate

In a 1-Liter, 4 neck kettle equipped with a thermocouple, a condenser, a nitrogen purge adapter, and a mechanical stirrer, 100 g of amber color viscous product from maleination reaction A1 was heated to 90° C. and added a mixture of 25.5 g (3 mole eq.) of calcium carbonate in 85 g (55 mole eq.) water over 3 hours controlling the foam formation. The reaction was held at 90° C. or 3 hours. The reaction yields >80% flowable light yellow color 60% active water-soluble product which was characterized by IR, and LC<0.05% residual maleic anhydride.

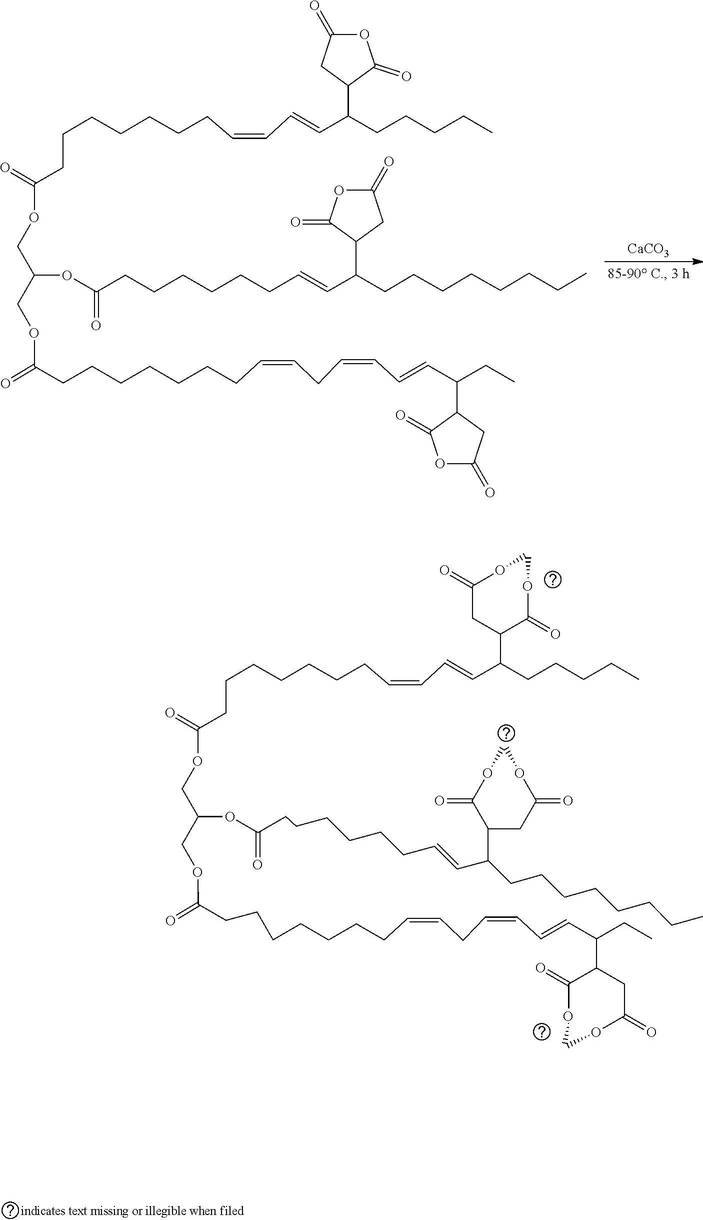

### Example F 1: Sodium Salt of Maleated Soybean Oil from Aqueous Sodium Hydroxide

In a 1-Liter, 4 neck kettle equipped with a thermocouple, a condenser, a nitrogen purge adapter, and a mechanical stirrer, 123 g of amber color viscous product from maleination reaction A1 is heated to 90° C. and added a mixture of 9.04 g (2 mole eq.) of 50% aqueous sodium hydroxide and 80 g (46 mole eq.) water over 1 hour controlling foaming and held for 3 hours. The reaction yields >96% flowable amber color 70% active water-soluble product which was characterized by IR, NMR and LC<0.05% residual maleic anhydride.

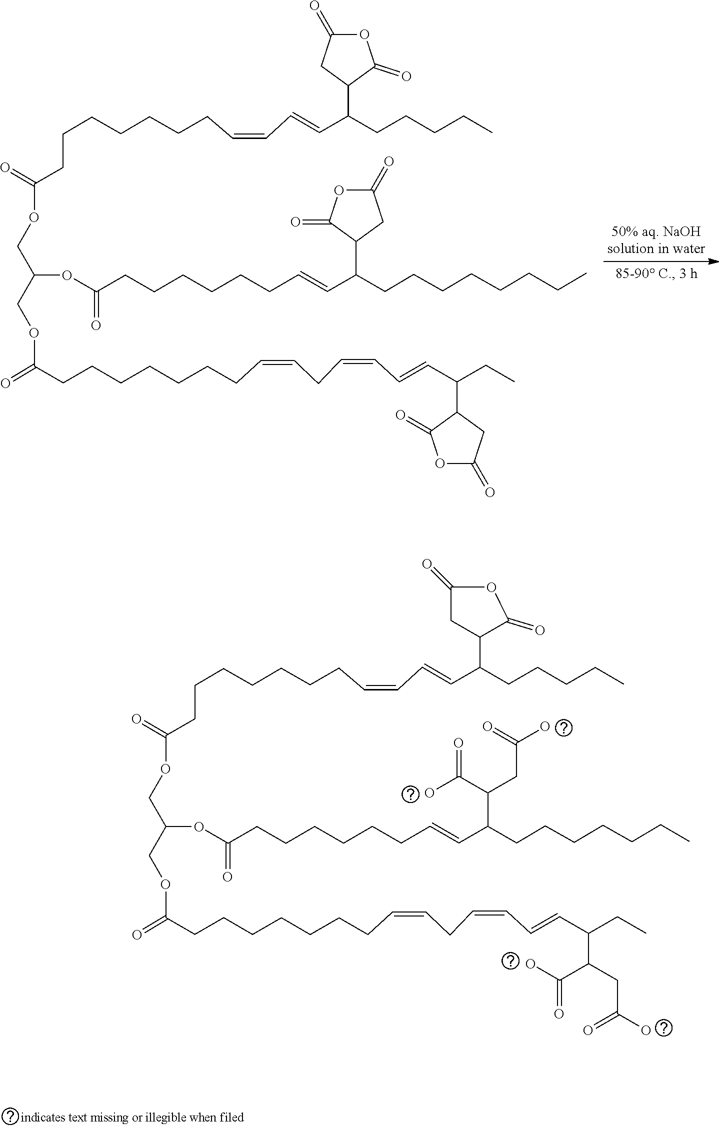 [SMILES: CCCCCCCCC(/C=C/CCCCCCC(=O)OC(COC(=O)CCCCCCC/C=C\C=C\C(CCCCC)C1CC(=O)OC1=O)COC(=O)CCCCCCC/C=C\C/C=C\C=C\C(CC)C1CC(=O)OC1=O)C1CC(=O)OC1=O ; CCCCCCCC(/C=C/CCCCCCC(=O)OC(COC(=O)CCCCCCC/C=C\C=C\C(CCCCC)C1CC(=O)OC1=O)COC(=O)CCCCCCC/C=C\C/C=C\C=C\C(CC)C(CC(=O)O)C(=O)O)C(CC(=O)O)C(=O)O]

### Example G 1: Sodium Salt of Maleated Soybean Oil Reacted with Hydrophobic Octyl Dodecanol

In a 1-Liter, 4 neck kettle equipped with a thermocouple, a condenser, a nitrogen purge adapter, and a mechanical stirrer, to the amber color viscous product from reaction F 1 and 34 g (1 mole eq.) of 2-octyl-1-dodecanol is mixed and heated to 90° C. and held for 8 hours. The one pot reaction without catalyst and solvent yields >90% amber color viscous product, which was characterized by NMR, IR, GC<5% for residual octyl dodecanol and LC<1% residual maleic anhydride.

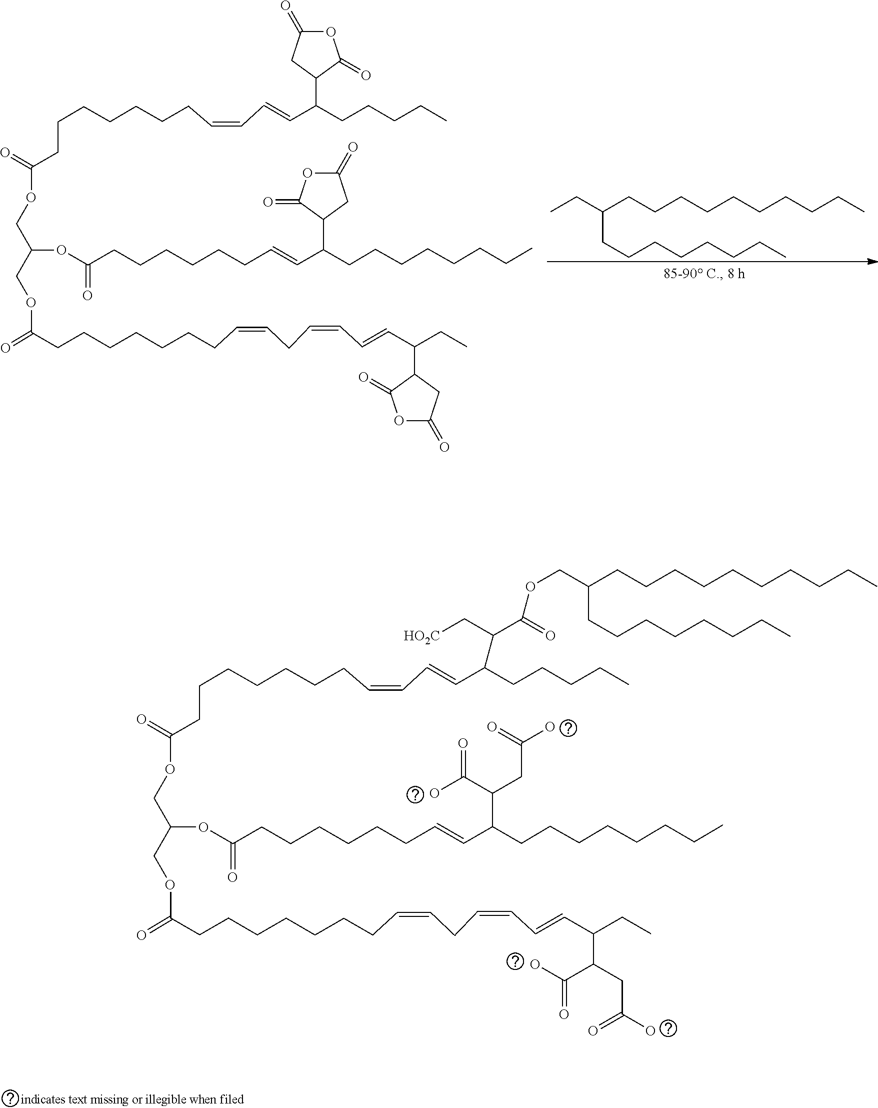

### Example H 1: Sodium Salt of Maleated Soybean Oil Reacted with Hydrophilic Glycerin

In a 1-Liter, 4 neck kettle equipped with a thermocouple, a condenser, a nitrogen purge adapter, and a mechanical stirrer, the amber color viscous product from reaction F 1 and 10.5 g (1 mole eq.) of glycerin was mixed and heated to 90° C. and held for 4-6 hours. The one pot reaction without catalyst and solvent yields >90% amber color viscous product which was characterized by NMR and LC<0.05% residual maleic anhydride.

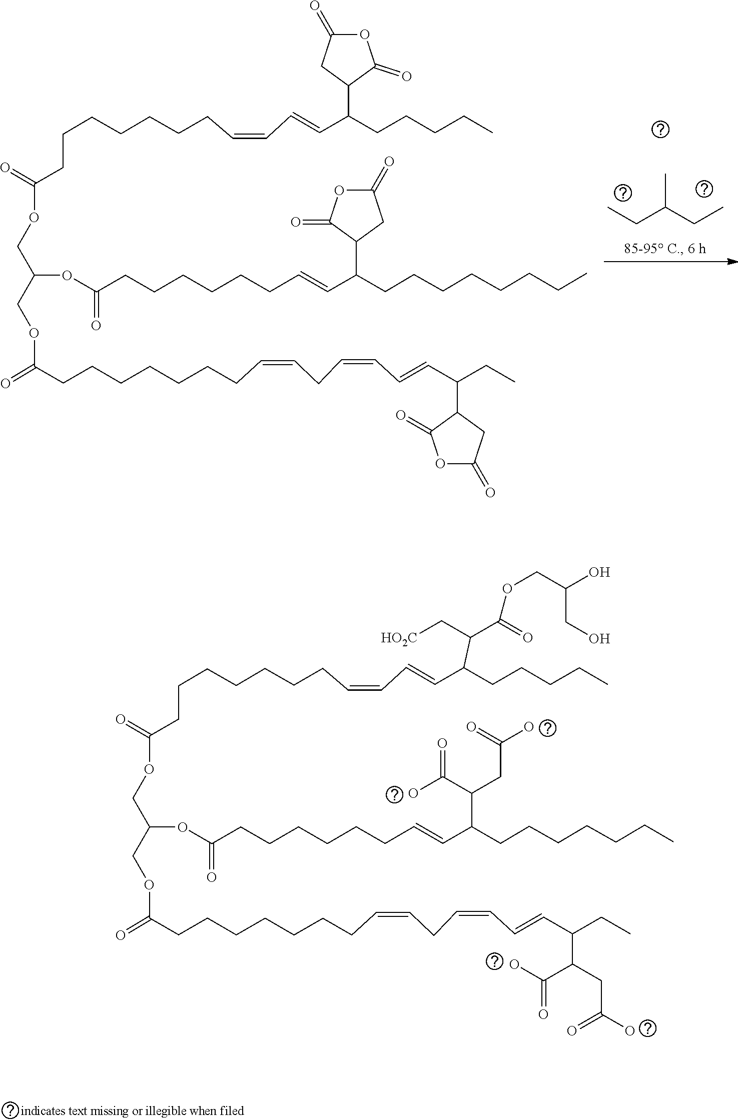

Toothpaste manufacture: Toothpaste formulations were made at the lab scale using methods and equipment commonly used by those skilled in the art. The procedure first involves forming the toothpaste gel phase (comprising the toothpaste rheology modifier gums in the toothpaste liquid phase), prior to adding the rest of the ingredients which are mixed under a vacuum of at least 28″ Hg in a double planetary mixer. The maleated soybean oil (3:1 molar substitution), sodium salt and the Gantrez S-97 copolymer (PVM/MA copolymer) toothpastes were made by dissolving these ingredients into the gel phase before processing under vacuum in the double planetary mixer.

Treatment of pellicle coated, hydroxyapatite discs and determination of active retention to the discs after challenge: Sintered, calcium deficient, hydroxyapatite (HAP) discs were coated with a mucin protein pellicle layer by treating the discs overnight at 37□ C. in a 1% mucin/electrolyte solution (‘artificial saliva’) before treatment. Toothpaste supernatants were prepared by making a 1:1 slurry of toothpaste in de-ionized water and centrifuging at 4,500×g for 45 minutes. The supernatant layer was poured off the top of the centrifuge tube and used as is.

Individual pellicle-coated HAP discs were placed into culture tubes containing the toothpaste supernatant solutions and shaken for 30-minutes at 37° C. in an incubating test tube shaker. The treated discs were then rinsed well in de-ionized water, placed into fresh culture tubes containing artificial saliva, and further challenged by vigorously shaking on the test tube shaker at 37° C. for 1-hour. The discs were then removed from the artificial saliva, rinsed in de-ionized water, and the compound of interest was extracted from the discs using a known weight of an organic solvent in which the compound was soluble. The concentrations of the compounds in the solvent extracts were determined using high performance liquid chromatography with UV detection.

### Example I: Essential Oil Toothpastes Used in Retention Study on Artificial Teeth

TABLE 1
Toothpaste compositions for essential oil containing toothpastes

| | Formula | Formula | Formula |
|---|---|---|---|
| | 1 | 2 | 3 |
| Ingredient | % (w/w) | % (w/w) | % (w/w) |
| Glycerin | 20.00 | 20.00 | 20.00 |
| Aqualon 7M8SF | 0.90 | 1.15 | 1.15 |
| Carboxymethyl cellulose | 0.30 | 0.30 | 0.30 |
| Sorbitol, 70% | 19.00 | 19.00 | 19.00 |
| Water | 31.56 | 32.51 | 34.51 |
| Sodium Fluoride | 0.243 | 0.243 | 0.243 |
| Sodium Saccharin | 0.30 | 0.30 | 0.30 |
| Sodium Hydroxide 50% | 1.20 | 0.00 | 0.00 |
| Gantrez S-97 copolymer | 2.00 | 0.00 | 0.00 |
| Maleated Soybean Oil, Sodium Salt | 0.00 | 2.00 | 0.00 |
| Zeodent 115 | 20.00 | 20.00 | 20.00 |
| Zeodent 165 | 1.50 | 1.50 | 1.50 |
| Titanium Dioxide | 0.50 | 0.50 | 0.50 |
| Essential Oil/Flavor Mixture (see | 1.00 | 1.00 | 1.00 |
| Table 2) | | | |
| Sodium Lauryl Sulfate | 1.50 | 1.50 | 1.50 |
| Totals: | 100.00 | 100.00 | 100.00 |
| Toothpaste pH (1:3 dispersion | 7.1 | 6.9 | 6.9 |
| in water): | | | |

TABLE 2
Essential oil/Flavor mixture

| Ingredient | % (w/w) |
|---|---|
| 4-Isopropyl m-cresol | 8.34 |
| IPMP | |
| Eugenol | 25.00 |
| Eucalyptol | 25.00 |
| Peppermint Oil | 41.66 |
| Total: | 100.00 |

### Example J: Triclosan Containing Toothpaste Used in Retention Study Artificial Teeth

TABLE 3
Toothpaste compositions for triclosan containing toothpastes

| | Formula | Formula | Formula |
|---|---|---|---|
| | 4 | 5 | 6 |
| Ingredient | % (w/w) | % (w/w) | % (w/w) |
| Glycerin | 20.00 | 20.00 | 20.00 |
| Aqualon CMC 7M8SF | 0.75 | 1.15 | 1.15 |
| Carrageenan | 0.30 | 0.30 | 0.30 |
| Sorbitol, 70% | 19.00 | 19.00 | 19.00 |
| Water | 31.41 | 32.21 | 34.21 |
| Sodium Fluoride | 0.243 | 0.243 | 0.243 |
| Sodium Saccharin | 0.30 | 0.30 | 0.30 |
| Sodium Hydroxide 50% | 1.20 | 0 | 0 |
| Gantrez S-97 copolymer | 2.00 | 0 | 0 |
| Maleated Soybean Oil (3:1), | 0 | 2.00 | 0 |
| Na-salt | | | |
| Zeodent 115 | 20.00 | 20.00 | 20.00 |
| Zeodent 165 | 1.50 | 1.50 | 1.50 |
| Titanium Dioxide, USP/FCC | 0.50 | 0.50 | 0.50 |
| Peppermint Oil, FCC | 1.00 | 1.00 | 1.00 |
| Sodium Lauryl Sulfate | 1.50 | 1.50 | 1.50 |
| Triclosan | 0.30 | 0.30 | 0.30 |
| Total | 100.00 | 100.00 | 100.00 |
| Toothpaste pH (1:3 dispersion | 7.2 | 7.0 | 7.2 |
| in water) | | | |

TABLE 4a
Mouthwash Formulation

| | % |
|---|---|
| Ingredient | (w/w) |
| Water | 85.93 |
| Sorbitol | 10 |
| Flavor | 0.5 |
| Cetylpyridinium chloride | 0.07 |
| Maleated Soybean oil Sodium salts | 1 |
| Poloxamer | 2 |
| Sweetener | 0.5 |
| Total | 100 |

TABLE 4b
Chewing gum Formulation

| Ingredient | % (w/w) |
|---|---|
| Gum base | 30 |
| Sorbitol | 66.8 |
| Glycerin | 0.5 |
| Maleated Soybean oil Sodium salts | 1 |
| Flavor | 1 |
| Sweetener | 0.5 |
| Lecithin | 0.2 |
| Total | 100 |

TABLE 5
Fast Dissolving Film

| | % |
|---|---|
| Ingredient | (w/w) |
| Pullulan | 93.68 |
| Sweetener | 1 |
| Menthol | 2 |
| Polysorbate 80 | 0.3 |
| Maleated Soybean oil Sodium salts | 2 |
| Color | 0.02 |
| Flavor | 1 |
| Total | 100 |

TABLE 6
Compressed Tablet

| Ingredient | % w/w |
|---|---|
| Xylitol, FCC | 57.00 |
| Microcrystalline cellulose | 10 |
| Sodium Bicarbonate | 9.76 |
| CMC | 3 |
| Maleated Soybean Oil Sodium salts | 1 |
| Sodium Fluoride | 0.24 |
| Hydrated silica | 10 |
| Sodium N-Cocoyl-L-Glutamate | 2 |
| Hydroxypropyl Cellulose | 3 |
| Sweetener | 1 |
| Powder Flavor | 2 |
| Magnesium Stearate | 1 |
| Total | 100 |

### Example K: In Vitro Actives Retention Experiments Using HAP Discs

Treatment of pellicle coated, hydroxyapatite discs and determination of active retention to the discs after challenge: Sintered, calcium deficient, hydroxyapatite (HAP) discs were coated with a mucin protein pellicle layer by treating the discs overnight at 37° C. in a 1% mucin/electrolyte solution (‘artificial saliva’) before treatment. Toothpaste supernatants were prepared by making a 1:1 slurry of toothpaste in de-ionized water and centrifuging at 4,500×g for 45 minutes. The supernatant layer was poured off the top of the centrifuge tube and used as is.

Individual pellicle-coated HAP discs (‘artificial teeth’) were placed into culture tubes containing the toothpaste supernatant solutions and shaken for 30-minutes at 37° C. in an incubating test tube shaker. The treated discs were then rinsed well in de-ionized water, placed into fresh culture tubes containing artificial saliva, and further challenged by vigorously shaking on the test tube shaker at 37° C. for 1-hour. The discs were then removed from the artificial saliva, rinsed in de-ionized water, and the compound of interest was extracted from the discs using a known weight of an organic solvent in which the compound was soluble. The concentrations of the compounds in the solvent extracts were determined using high performance liquid chromatography with UV detection.

Table 7 shows the retention results of essential oils using pellicle coated HAP discs treated with the supernatants of centrifuged 1:1 slurrys of toothpaste in water. The toothpaste formulae are shown in Example I; with Formula 1 made with 2% Gantrez S-97 copolymer, Formula 2 made with 2% of the maleated soybean oil, sodium salt and Formula 3 containing no retention enhancing ingredient (negative control). The essential oils were extracted from the artificial teeth using absolute ethanol and the extracts were assayed for each compound using an HPLC method with UV detection. Each data point is the average of three replicates.

TABLE 7
Retention of essential oils on pellicle coated HAP discs

| | Formula 1 | | Formula 2 | | Formula 3 | |
|---|---|---|---|---|---|---|
| | (μg/disc) | SD | (μg/disc) | SD | (μg/disc) | SD |
| IPMP | 5.20 | 0.44 | 7.33 | 0.15 | 3.80 | 0.10 |
| recovery | | | | | | |
| Eugenol | 7.60 | 0.72 | 9.80 | 0.44 | 5.70 | 0.00 |
| recovery | | | | | | |

Table 8 shows the retention results of triclosan from pellicle coated HAP discs treated with the supernatants of centrifuged 1:1 slurrys of toothpaste in water. The toothpaste formulae are shown in Example J; with Formula 4 made with 2% Gantrez S-97 copolymer, Formula 5 made with 2% of the maleated soybean oil, sodium salt and Formula 6 containing no retention enhancing ingredient (negative control). Triclosan was extracted from the artificial teeth using acetonitrile and the extracts were assayed for triclosan using an HPLC method with UV detection. Each data point is the average of three replicates.

TABLE 8

| Retention | | | | | | |
|---|---|---|---|---|---|---|
| of triclosan | | | | | | |
| on pellicle | Formula | | Formula | | Formula | |
| coated | 4 | | 5 | | 6 | |
| HAP discs | (μg/disc) | SD | (μg/disc) | SD | (μg/disc) | SD |
| Triclosan | 35.77 | 6.65 | 63.87 | 1.74 | 25.60 | 0.28 |
| Recovery | | | | | | |

While the compositions and methods of the disclosed and/or claimed inventive concept(s) have been described in terms of particular aspects, it will be apparent to those of ordinary skill in the art that variations may be applied to the compositions and/or methods and in the steps or in the sequence of steps of the method described herein without departing from the concept, spirit and scope of the disclosed and/or claimed inventive concept(s). All such similar substitutes and modifications apparent to those skilled in the art are deemed to be within the spirit, scope and concept of the disclosed and/or claimed inventive concept(s).

We claim:

1. An oral care composition comprising a reaction product of
(a) a maleated natural oil, comprising a natural oil with maleated functionality; and
(b) a base.

2. The oral care composition according to claim 1, wherein the reaction product comprises a maleated functionality fully or partially reacted with the base.

3. The oral care composition according to claim 1, wherein the base is selected from the group consisting of inorganic bases, organic bases, and mixtures thereof.

4. The oral care composition according to claim 3, wherein the inorganic base is selected from the group consisting of oxides of alkali metals and alkaline earth metals, hydroxides of alkali metals and alkaline earth metals, carbonates of alkali metals and alkaline earth metals, bicarbonates of alkali metals and alkaline earth metals, oxides of transition metals, and combinations thereof.

5. The oral care composition according to claim 3, wherein the organic base is selected from the group consisting of ammonia, primary amines, secondary amines, pyridine, imidazole, benzimidazole, histidine, guanidine, and mixtures thereof.

6. The oral care composition according to claim 4, wherein the alkali metal is selected from the group consisting of lithium, sodium, potassium, rubidium, cesium, francium, and mixtures thereof.

7. The oral care composition according to claim 4, wherein the alkaline earth metal is selected from the group consisting of beryllium, magnesium, calcium, strontium, barium, radium, and mixtures thereof.

8. The oral care composition according to claim 4, wherein the transition metal is selected from the group consisting of oxides, hydroxides, carbonates or bicarbonates of scandium, titanium, vanadium, chromium, manganese, iron, cobalt, nickel, copper, zinc, yttrium zirconium, niobium, molybdenum, technetium, ruthenium, rhodium, palladium, silver, cadmium, hafnium, tantalum, tungsten, rhenium, osmium, iridium, platinum, gold, mercury, rutherfordium, dubnium, seaborgium, bohrium, hassium, meitnerium, darmstadtium, roentgenium, and mixtures thereof.

9. The oral care composition according to claim 4, wherein the inorganic base is selected from the group consisting of oxides, hydroxides, carbonates and bicarbonates of sodium, calcium, and combinations thereof.

10. The oral care composition according to claim 1, wherein the maleated natural oil is selected from the group consisting of maleated avocado oils, maleated coconut oils, maleated corn oils, maleated cottonseed oils, maleated jojoba oils, maleated linseed oils, maleated nut oils, maleated olive oils, maleated palm oils, maleated raisin oils, maleated rapeseed oils, maleated safflower oils, maleated sesame oils, maleated soybean oils, maleated squash oils, maleated sunflower oils, maleated almond oils, maleated canola oils, maleated flaxseed oils, maleated grapeseed oils, maleated palm oils, maleated palm kernel oils, maleated peanut oils, maleated walnut oils, and mixture thereof.

11. The oral care composition according to claim 1, wherein the maleated natural oil is a maleated soybean oil and the base is sodium hydroxide.

12. The oral care composition according to claim 1, wherein the reaction product is selected from the group of structures, comprising a maleated functionality fully or partially reacted with the base, consisting of the following structures: 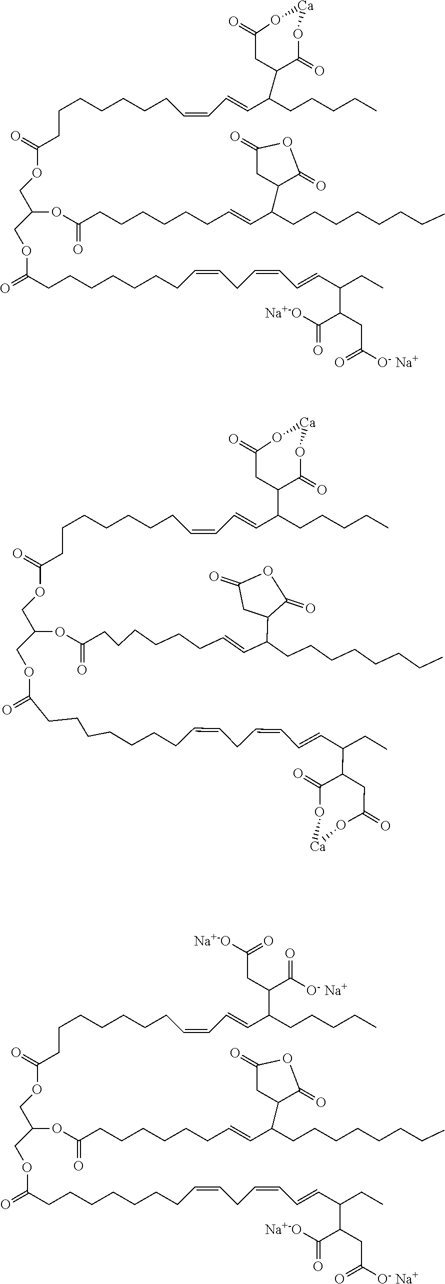 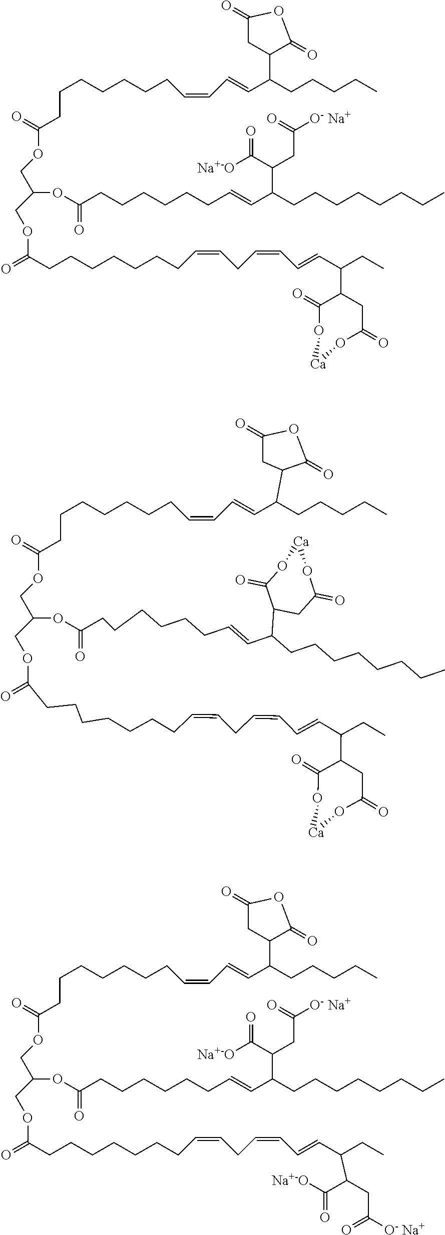 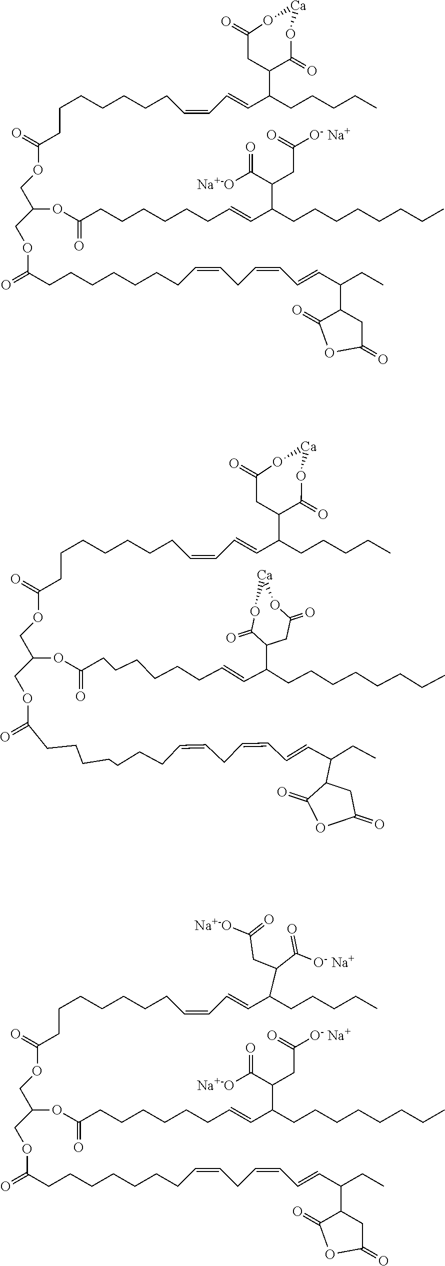 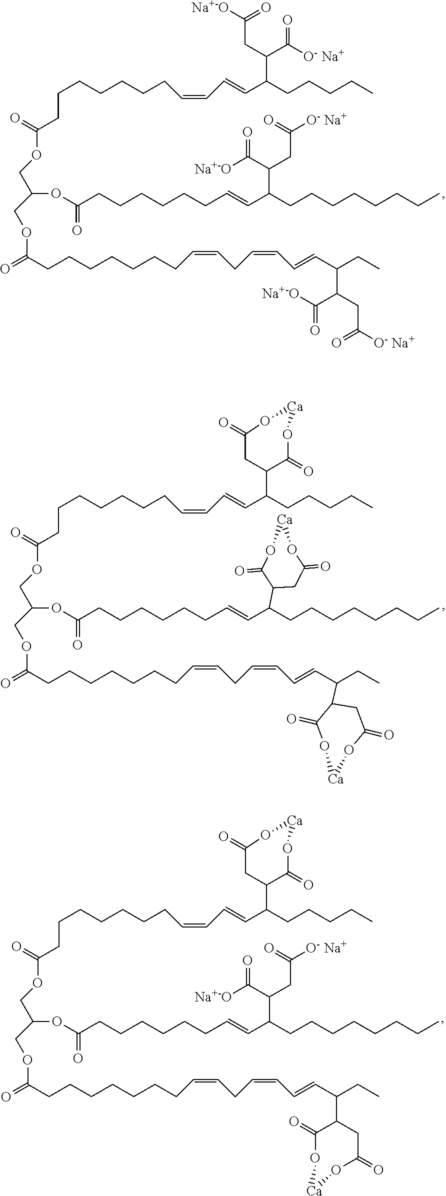
and mixtures thereof.

13. The oral care composition according to claim 1, wherein the reaction product further comprises an orally acceptable excipient and an oral care active ingredient.

14. The oral care composition according to claim 13, wherein the reaction product comprises:
(A) (a) from about 0.1 to about 99.9 wt. % of a maleated natural oil, comprising a natural oil with maleated functionality; and (b) a base, and
(B) (a) from about 0.1 to about 99.9 wt. % of an orally acceptable excipient; and
(b) from about 0.1 to about 50 wt. % of an oral care active ingredient.

15. The oral care composition according to claim 14, wherein the reaction product comprises:
(A) (a) from about 0.1 to about 99.9 wt. % of a maleated soybean natural oil, comprising a soybean oil with maleated functionality; and (b) a base selected from the group consisting of sodium hydroxide, potassium hydroxide, calcium hydroxide; and mixtures thereof;
(B) (a) from about 0.1 to about 99.9 wt. % of an orally acceptable excipient; and
(b) from about 0.1 to about 50 wt. % of an oral care active ingredient.

16. An oral care composition comprising:
(A) a reaction product of:
(a) a maleated natural oil, comprising a natural oil with maleated functionality; and
(b) a base;
wherein the reaction product comprises a maleated functionality partially reacted with the base; and
(B) reacting the reaction product from (A) with:
a functionalized or unfunctionalized moiety selected from the group consisting of hydrophobic moieties, hydrophilic moieties, and combinations thereof.

17. The oral care composition according to claim 16, wherein the hydrophobic moiety is a moiety selected from the group consisting of unsubstituted or substituted alkyl, cycloalkyl, alkenyl, and aryl alcohols, wherein any of the beforehand mentioned groups may be with or without heteroatoms, containing from about C₆ to about C₃₆ atoms, unsubstituted or substituted alkyl, cycloalkyl, alkenyl, and aryl amines, wherein any of the beforehand mentioned groups may be with or without heteroatoms, containing from about C₆ to about C₃₆ atoms, silicon-based compounds, and combinations thereof.

18. The oral care composition according to claim 16, wherein the hydrophilic moiety is a moiety selected from the group consisting of unsubstituted or substituted alkyl, cycloalkyl, alkenyl, and aryl alcohols, wherein any of the beforehand mentioned groups may be with or without heteroatoms, containing from about C₁ to about C₅ atoms, unsubstituted or substituted alkyl, cycloalkyl, alkenyl, and aryl amines, wherein any of the beforehand mentioned groups may be with or without heteroatoms, containing from about C₁ to about C₅ atoms, unsubstituted or substituted polyols, wherein any of the beforehand mentioned groups may be with or without heteroatoms, containing from about C₂ to about C₃₆ atoms, silanes, and combinations thereof.

19. The oral care composition according to claim 18, wherein the silane is functionalized with an alcohol or an amine, and combinations thereof.

20. The oral care composition according to claim 17, wherein the hydrophobic alcohol is selected from the group consisting of heptanol, nonanol, decanol, dodecanol, phenol, ethylbenzyl alcohol, 2-ethyl-1-hexanol, 1-octanol, 2-octanol, 2-octyl-1-dodecyl alcohol, 2-tertradecanol, 2-hexadecanol, 3,7-dimethyl-1-octanol, 2-propyl-1-pentanol, 4-methyl-1-pentanol, and mixtures thereof.

21. The oral care composition according to claim 18, wherein the hydrophilic alcohol is selected from the group consisting of methanol, ethanol, propanol, isopropanol, butanol, ethylene glycol, propylene glycol, diethylene glycol, dipropylene glycol, dibutylene glycol, polyethylene glycol, methoxypolyethylene glycol, polypropylene glycol, hexylene glycol, sorbitol, neopentylglycol, eythritol, mannitol, xylitol, threitol, pentaerythritol, beta-cyclodextrin, L-Ribose, 2-deoxy-D-galactose, and mixtures thereof.

22. The oral care composition according to claim 17, wherein the hydrophobic amine is selected from the group consisting of benzylamine, cyclohexylamine, hexylamine, methylhexylamine, phenethylamine, octylamine, oleylamine, decylamine, dodecylamine, octadecylamine, undecyl amine, pentadecyl amine, 2-methyl butyl amine, dimethyl amine, and mixtures thereof.

23. The oral care composition according to claim 18, wherein the hydrophilic amine is selected from the group consisting of 2-methylpentane-1,5-diamine, diethanol amine, diisopropanolamine, serinol hydrochloride, 2-amino-2-ethyl-1,3-propanediol, n-methyl-D-glucamine, D-galactosamine hydrochloride, d-glucosamine hydrochloride, D-mannosamine hydrochloride, and mixtures thereof.

24. The oral care composition according to claim 17, wherein the silicon based compound is a hydrophobic compound selected from the group consisting of aminopropylmethylsiloxane-dimethylsiloxane, N-ethylaminoisobutyl terminated polydimethylsiloxane, poly(1,1-dimethylsilazane) telomer, aminopropyl terminated polydimethylsiloxane, monoaminopropyl terminated polydimethylsiloxane, (tetramethylpiperidinyloxy)propylmethylsiloxane]-dimethylsiloxane copolymer, polydimethylsiloxane, carbinol (hydroxyl) terminated polydimethylsiloxane, monocarbinol terminated polydimethylsiloxane, monocarbinol terminated functional polydimethylsiloxane, [Bis(hydroxyethyl)amine] terminated polydimethylsiloxane, silanol terminated polydimethylsiloxane, silanol terminated polydiphenylsiloxane, dodecylmethylsiloxane-hydroxypolyalkyleneoxypropyl methylsiloxane, and mixtures thereof.

25. The oral care composition according to claim 18, wherein the silane is a hydrophilic compound selected from the group consisting of 3-aminopropylsilanetriol, N-(2-aminoethyl)-3-aminopropylsilanetriol, and mixtures thereof.

26. The oral care composition according to claim 16, wherein the maleated natural oil is selected from the group consisting of maleated avocado oils, maleated coconut oils, maleated corn oils, maleated cottonseed oils, maleated jojoba oils, maleated linseed oils, maleated nut oils, maleated olive oils, maleated palm oils, maleated raisin oils, maleated rapeseed oils, maleated safflower oils, maleated sesame oils, maleated soybean oils, maleated squash oils, maleated sunflower oils, maleated almond oils, maleated canola oils, maleated flaxseed oils, maleated grapeseed oils, maleated palm oils, maleated palm kernel oils, maleated peanut oils, maleated walnut oils, and mixtures thereof.

27. The oral care composition according to claim 26 wherein the maleated natural oil is a maleated soybean oil.

28. The oral care composition according to claim 16, wherein the reaction product is selected from the group of structures represented by the following structures: 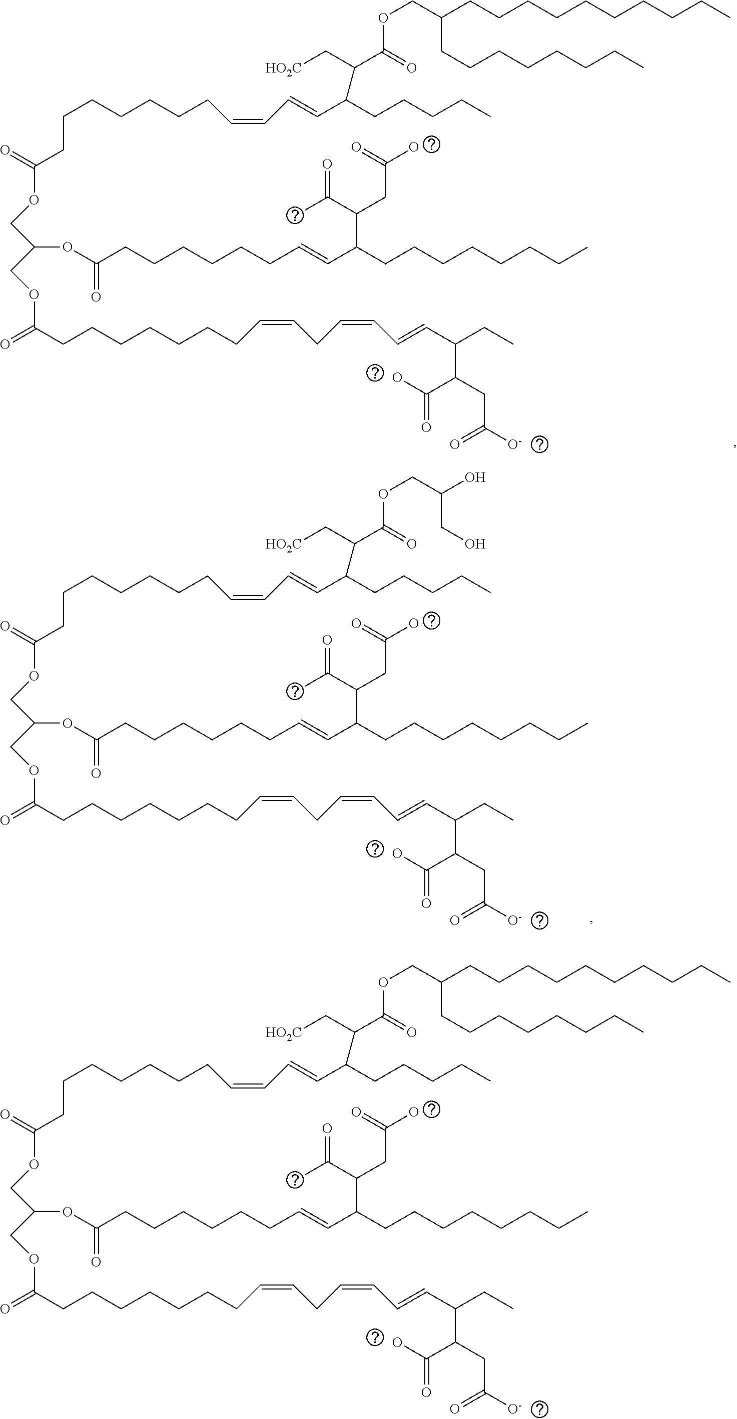 [SMILES: CCCCCCCCCCC(CCCCCCCC)COC(=O)C(CC(=O)O)C(/C=C/C=C\CCCCCCCC(=O)OCC(COC(=O)CCCCCCC/C=C\C/C=C\C=C\C(CC)C(CC(=O)[O-])C(=O)O)OC(=O)CCCCCC/C=C/C(CCCCCCCC)C(CC(=O)O)C(C)=O)CCCCC ; CCCCCCCCC(/C=C/CCCCCCC(=O)OC(COC(=O)CCCCCCC/C=C\C=C\C(CCCCC)C(CC(=O)O)C(=O)OCC(O)CO)COC(=O)CCCCCCC/C=C\C/C=C\C=C\C(CC)C(CC(=O)[O-])C(=O)O)C(CC(=O)O)C(C)=O ; CCCCCCCCCCC(CCCCCCCC)COC(=O)C(CC(=O)O)C(/C=C/C=C\CCCCCCCC(=O)OCC(COC(=O)CCCCCCC/C=C\C/C=C\C=C\C(CC)C(CC(=O)[O-])C(=O)O)OC(=O)CCCCCC/C=C/C(CCCCCCCC)C(CC(=O)O)C(C)=O)CCCCC] 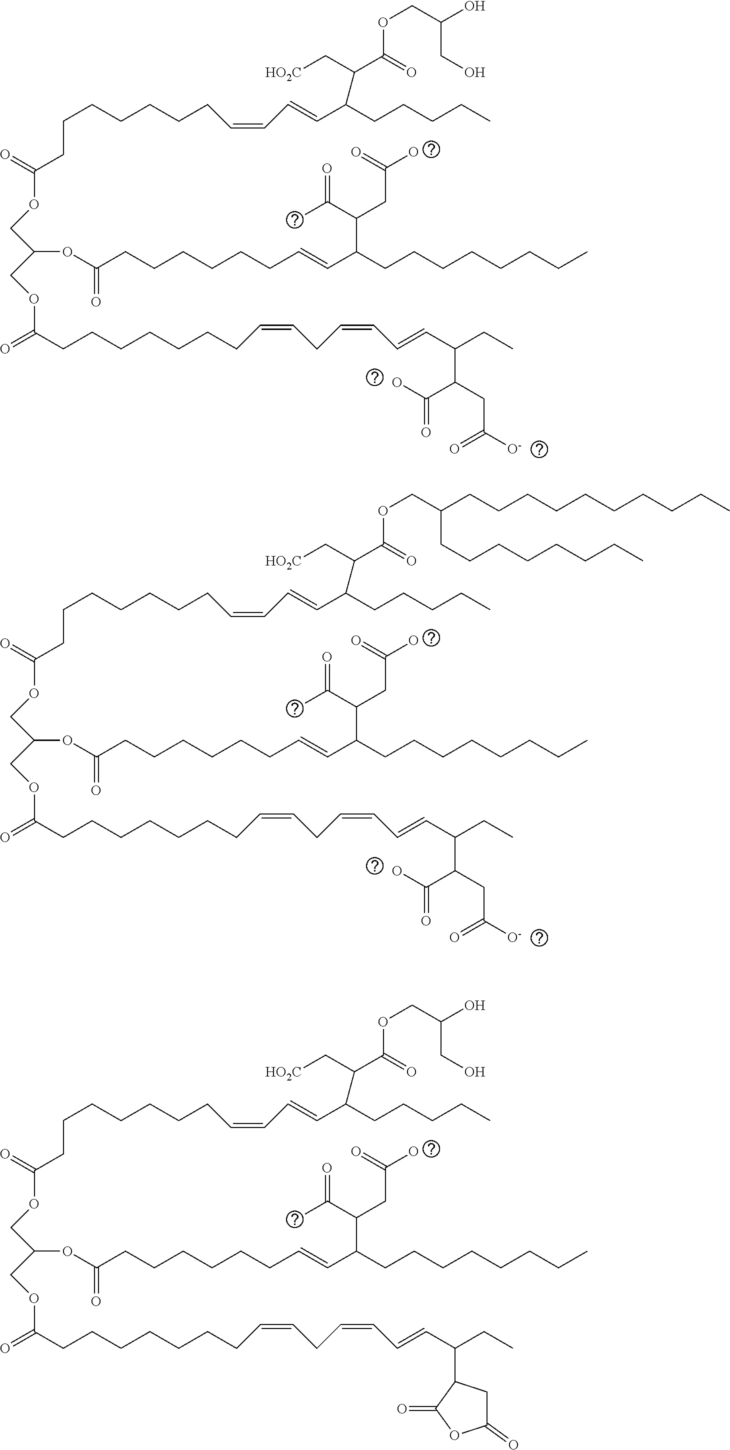 [SMILES: CCCCCCCCC(/C=C/CCCCCCC(=O)OC(COC(=O)CCCCCCC/C=C\C=C\C(CCCCC)C(CC(=O)O)C(=O)OCC(O)CO)COC(=O)CCCCCCC/C=C\C/C=C\C=C\C(CC)C(CC(=O)[O-])C(=O)O)C(CC(=O)O)C(C)=O ; CCCCCCCCCCC(CCCCCCCC)COC(=O)C(CC(=O)O)C(/C=C/C=C\CCCCCCCC(=O)OCC(COC(=O)CCCCCCC/C=C\C/C=C\C=C\C(CC)C(CC(=O)[O-])C(=O)O)OC(=O)CCCCCC/C=C/C(CCCCCCCC)C(CC(=O)O)C(C)=O)CCCCC ; CCCCCCCCC(/C=C/CCCCCCC(=O)OC(COC(=O)CCCCCCC/C=C\C=C\C(CCCCC)C(CC(=O)O)C(=O)OCC(O)CO)COC(=O)CCCCCCC/C=C\C/C=C\C=C\C(CC)C1CC(=O)OC1=O)C(CC(=O)O)C(C)=O] 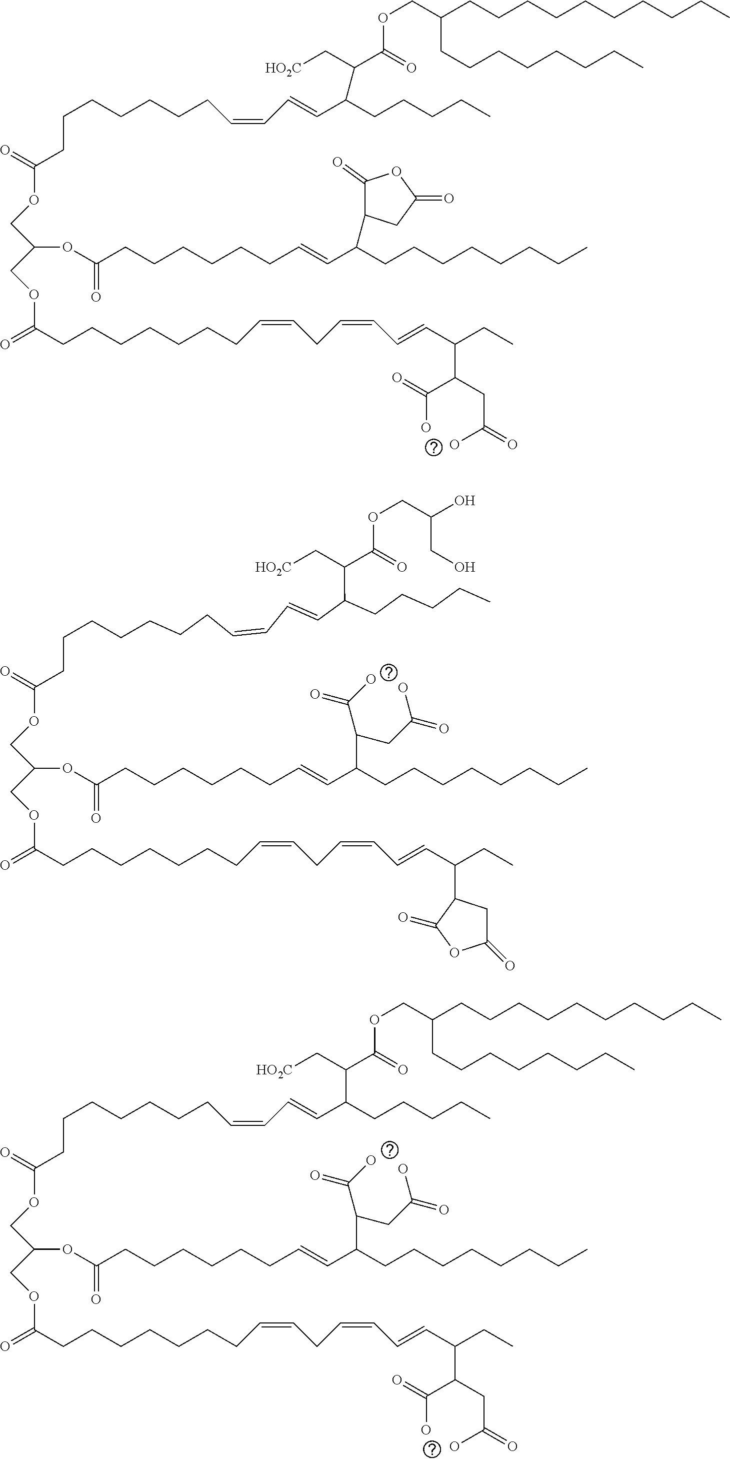 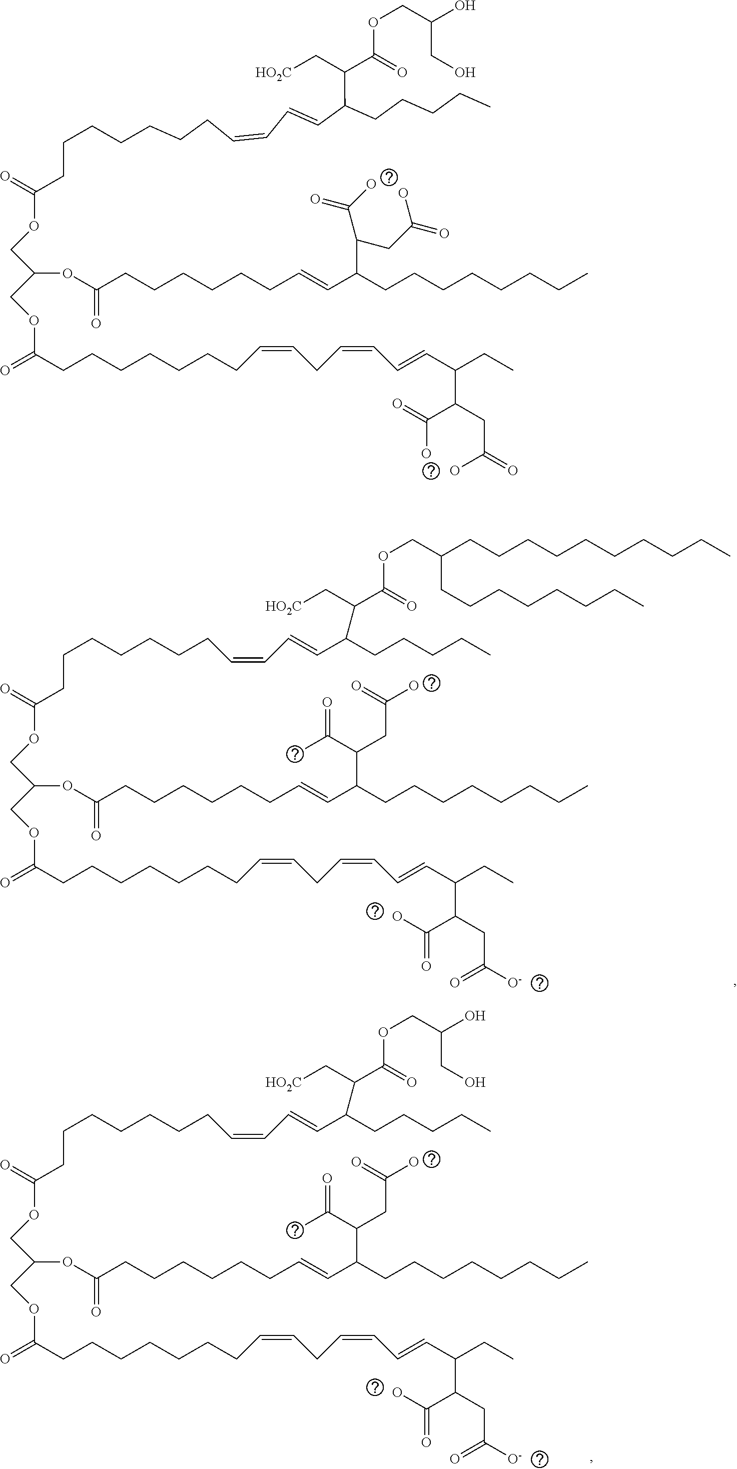 [SMILES: CCCCCCCCC(/C=C/CCCCCCC(=O)OC(COC(=O)CCCCCCC/C=C\C=C\C(CCCCC)C(CC(=O)O)C(=O)OCC(O)CO)COC(=O)CCCCCCC/C=C\C/C=C\C=C\C(CC)C(CC(=O)O)C(=O)O)C(CC(=O)O)C(=O)O ; CCCCCCCCCCC(CCCCCCCC)COC(=O)C(CC(=O)O)C(/C=C/C=C\CCCCCCCC(=O)OCC(COC(=O)CCCCCCC/C=C\C/C=C\C=C\C(CC)C(CC(=O)[O-])C(=O)O)OC(=O)CCCCCC/C=C/C(CCCCCCCC)C(CC(=O)O)C(C)=O)CCCCC ; CCCCCCCCC(/C=C/CCCCCCC(=O)OC(COC(=O)CCCCCCC/C=C\C=C\C(CCCCC)C(CC(=O)O)C(=O)OCC(O)CO)COC(=O)CCCCCCC/C=C\C/C=C\C=C\C(CC)C(CC(=O)[O-])C(=O)O)C(CC(=O)O)C(C)=O] 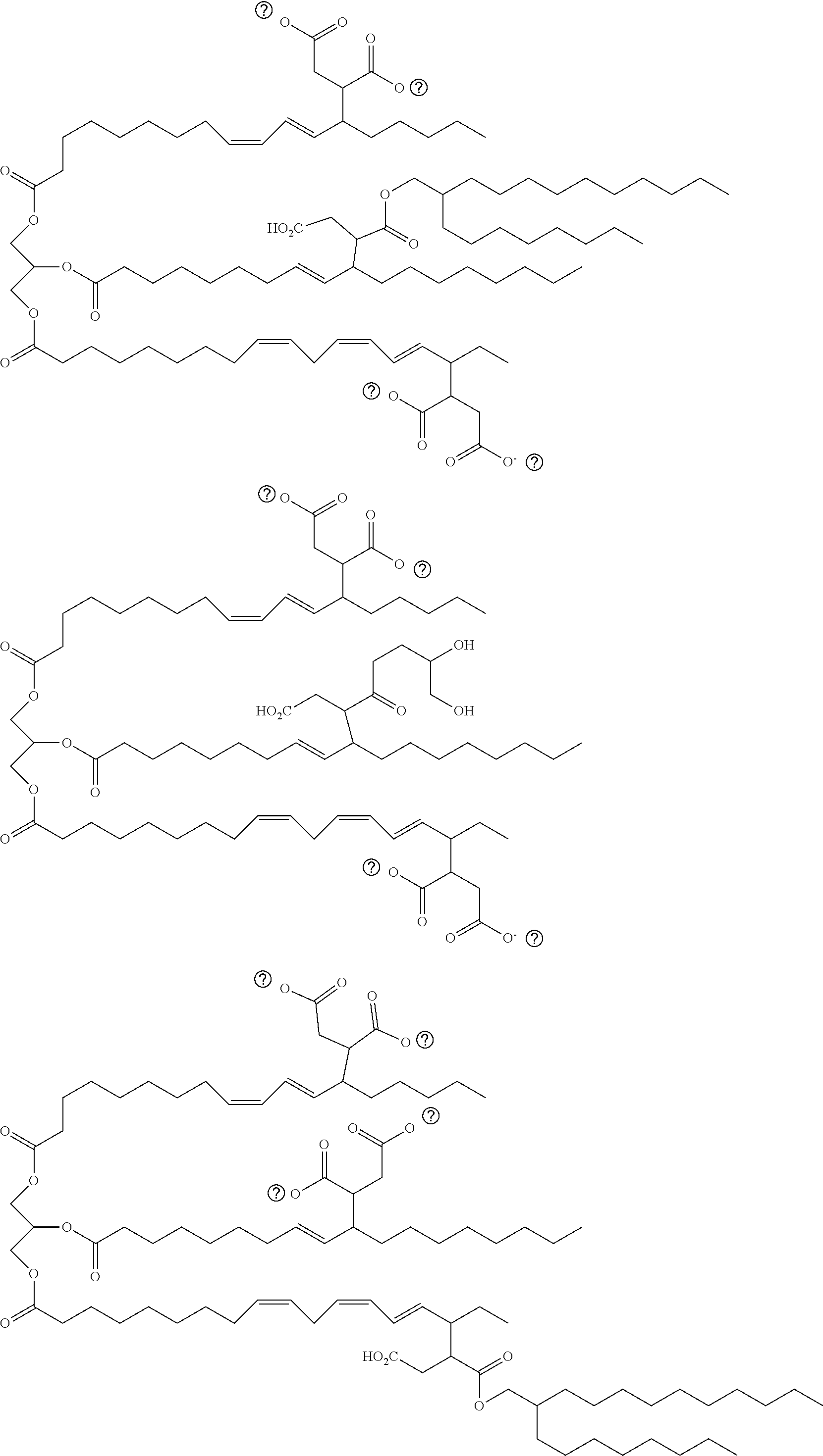 [SMILES: CCCCCCCCCCC(CCCCCCCC)COC(=O)C(CC(=O)O)C(/C=C/CCCCCCC(=O)OC(COC(=O)CCCCCCC/C=C\C=C\C(CCCCC)C(CC(=O)O)C(=O)O)COC(=O)CCCCCCC/C=C\C/C=C\C=C\C(CC)C(CC(=O)[O-])C(=O)O)CCCCCCCC ; CCCCCCCCC(/C=C/CCCCCCC(=O)OC(COC(=O)CCCCCCC/C=C\C=C\C(CCCCC)C(CC(=O)O)C(=O)O)COC(=O)CCCCCCC/C=C\C/C=C\C=C\C(CC)C(CC(=O)[O-])C(=O)O)C(CC(=O)O)C(=O)CCC(O)CO ; CCCCCCCCCCC(CCCCCCCC)COC(=O)C(CC(=O)O)C(/C=C/C=C\C/C=C\CCCCCCCC(=O)OCC(COC(=O)CCCCCCC/C=C\C=C\C(CCCCC)C(CC(=O)O)C(=O)O)OC(=O)CCCCCC/C=C/C(CCCCCCCC)C(CC(=O)O)C(=O)O)CC] 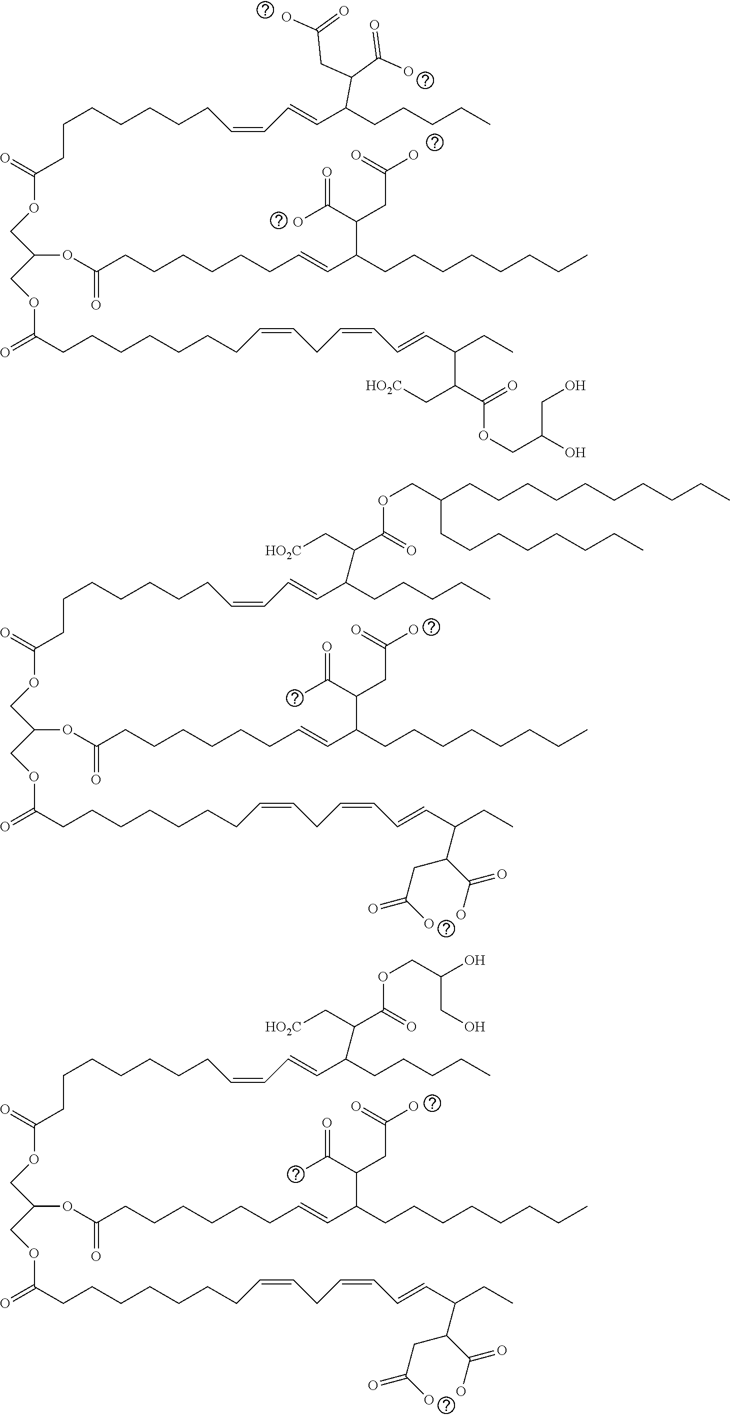 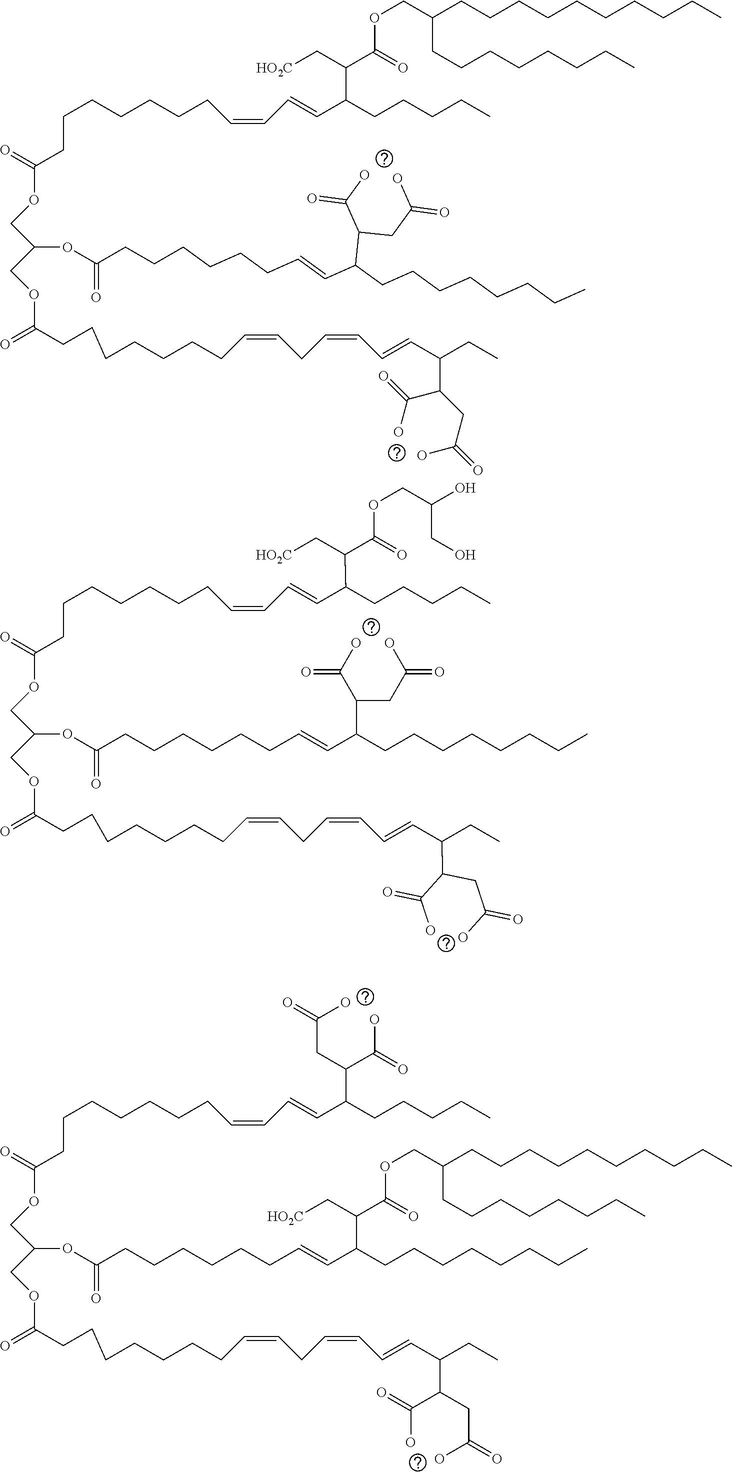 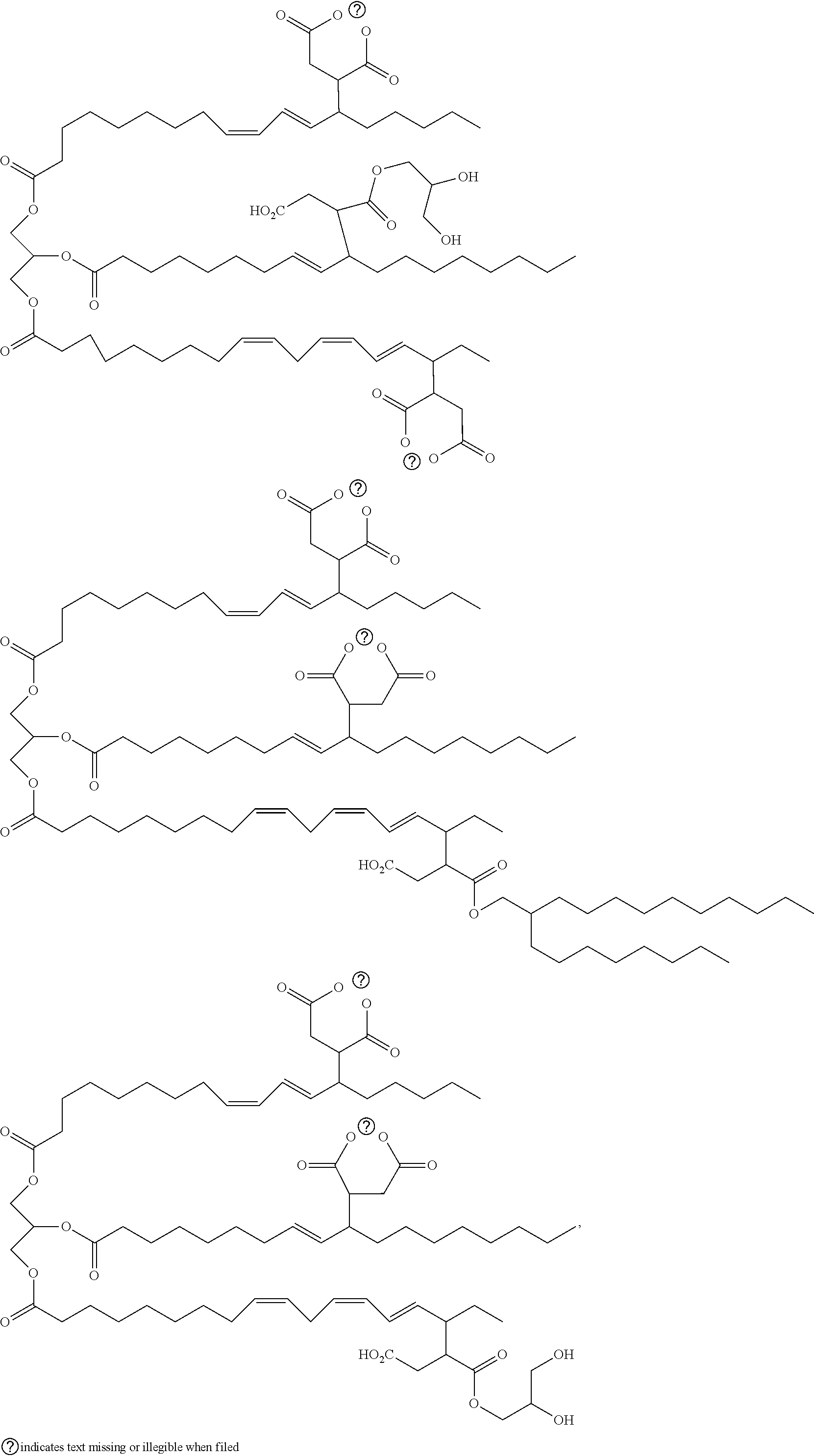 [SMILES: CCCCCCCCC(/C=C/CCCCCCC(=O)OC(COC(=O)CCCCCCC/C=C\C=C\C(CCCCC)C(CC(=O)O)C(=O)O)COC(=O)CCCCCCC/C=C\C/C=C\C=C\C(CC)C(CC(=O)O)C(=O)O)C(CC(=O)O)C(=O)OCC(O)CO ; CCCCCCCCCCC(CCCCCCCC)COC(=O)C(CC(=O)O)C(/C=C/C=C\C/C=C\CCCCCCCC(=O)OCC(COC(=O)CCCCCCC/C=C\C=C\C(CCCCC)C(CC(=O)O)C(=O)O)OC(=O)CCCCCC/C=C/C(CCCCCCCC)C(CC(=O)O)C(=O)O)CC ; CCCCCCCCC(/C=C/CCCCCCC(=O)OC(COC(=O)CCCCCCC/C=C\C=C\C(CCCCC)C(CC(=O)O)C(=O)O)COC(=O)CCCCCCC/C=C\C/C=C\C=C\C(CC)C(CC(=O)O)C(=O)OCC(O)CO)C(CC(=O)O)C(=O)O]
and combinations thereof.

29. The oral care composition according to claim 16, wherein the composition is formulated as a toothpaste, tooth gel, subgingival gel, tooth powder, mouth-rinse, mouth wash, mousse, foam, denture product, mouth-spray, chewable tablet, dissolvable film, chewing gum, and mixtures thereof.

30. The oral care composition according to claim 16, wherein the composition is an aqueous or non-aqueous based end-user composition.

31. The oral care composition according to claim 16, wherein the pH of the composition is in the range from about 4 to about 10.

32. The oral care composition according to claim 16, wherein the composition further comprises an orally acceptable excipient selected from the group consisting of thickening agents, desensitizing agents, whitening agents, tartar control agents, abrasives, binders, detergents, adhesion agents, foam modulators, pH modifying agents, mouth feel agents, sweeteners, flavorants, colorants, preservatives, humectants, fluoride containing salts and compounds, water, polymers, orally acceptable surfactants, and mixtures thereof.

33. The oral care composition according to claim 16, wherein the composition further comprises an oral care active ingredient selected from the group consisting of an analgesic, an antibacterial, an antibiotic, a probiotic, an antioxidant, a peptide, an enzyme, a cooling agent, and mixtures thereof.

34. A compound having the following structure: [chemical structure]
